(19) United States
(12) Patent Application Publication
(10) Pub. No.: US 2023/0212321 A1
(43) Pub. Date: Jul. 6, 2023

(54) METHODS AND MEANS FOR THE PRODUCTION OF IG-LIKE MOLECULES

(71) Applicant: MERUS N.V., Utrecht (NL)

(72) Inventors: Cornelis DE KRUIF, Utrecht (NL); Linda HENDRIKS, Utrecht (NL)

(21) Appl. No.: 17/926,283

(22) PCT Filed: May 20, 2021
(86) PCT No.: PCT/NL2021/050322

§ 371 Date: Nov. 18, 2022

(30) Foreign Application Priority Data
May 21, 2020 (EP) 20175903.2

(51) Int. Cl. C07K 16/46

(52) U.S. Cl. CPC C07K 16/468; C07K 2317/526; C07K 2317/21; C07K 2317/94; C07K 2317/31

(57) ABSTRACT

The invention relates to heterodimeric proteins, and their methods of production and collection, that are useful to treat human disease.

The invention relates to the fields of molecular biology, medicine and biological therapeutics. It particularly relates to the field of therapeutic antibodies for the treatment of various diseases.

### BACKGROUND

During the past decade, bispecific antibodies have emerged as an alternative to the use of combinations of two antibodies. Similarly, multivalent multimers, capable of binding three or more of the same or different antigens or epitopes have emerged as a developing field of technology. Whereas a combination of two antibodies represents a mixture of two different immunoglobulins that bind to different epitopes on the same or different targets, in a bispecific antibody this is achieved through a single immunoglobulin. In a multivalent multimer, binding to three or more different epitopes on the same or different targets may be achieved.

By binding to two epitopes on the same or different targets, bispecific antibodies may have similar effects or superior effects as compared to a combination of two antibodies binding to the same epitopes. This may also apply to the generation of multivalent multimers, capable of binding three or more targets. Furthermore, since bispecific antibodies of the IgG format combine two different monovalent binding regions in a single molecule and mixtures of two IgG antibodies combine two different bivalent binding molecules in a single preparation, and multivalent multimers may combine three or more binding regions, different effects of these formats have been observed as well. From a technological and regulatory perspective, if these bispecific or multivalent multimers can be efficiently generated and separated in a substantially pure manner, this makes development of a single bispecific antibody or multivalent multimer less complex because manufacturing, preclinical and clinical testing involve a single molecule. Thus, therapies based on a single bispecific antibody or multivalent multimer are facilitated by a less complicated and cost-effective drug development process while providing more efficacious antibody therapies.

Bispecific antibodies based on the IgG format, consisting of two heavy and two light chains have been produced by a variety of methods. For instance, bispecific antibodies may be produced by fusing two antibody-secreting cell lines to create a new cell line or by expressing two antibodies in a single cell using recombinant DNA technology. These approaches yield multiple antibody species as the respective heavy chains from each antibody may form monospecific dimers (also called homodimers), which contain two identical paired heavy chains with the same specificity, and bispecific dimers (also called heterodimers) which contain two different paired heavy chains with different specificity, and unpaired heavy or light chains referred to as half bodies may result in a mixture of cell products. In addition, light chains and heavy chains from each antibody may randomly pair to form non-functional combinations. This problem, known as heavy and light chain miss-pairings, can be addressed by choosing antibodies that share a common light chain for expression as a bispecific. Recently, applicants have shown the capability of generating a multivalent multimer comprising three or more heavy and three or more light chains, where either heavy or light, but preferably light is a common chain.

But even when a common light chain is used, expression of two or more heavy chains that contain variations that drive heterodimer formation and one common light chain in a single cell may result in three different antibody species and halfbody impurities. Expression of heavy chains having engineered variations of residues in the Fc may promote heterodimer formation, yet still may generate residual halfbodies, homodimers or generate heterodimers that have lower stability due to the engineered variations. Expression of a bispecific antibody may still result in expression of two monospecific ‘parental’ antibodies and the bispecific antibody containing the matched Fc regions. The more halfbodies and/or monospecific antibodies formed, the lower the overall efficiency of the cell expression system, and potentially more costly and time consuming processes are required to separate the bispecific or multivalent multimer of interest. Accordingly, if a single bispecific or multivalent multimer product is desired, the bispecific antibody or multivalent multimer of interest needs to be purified from the resulting mixture.

The need to recover the bispecific antibody or multivalent multimer is important in the context of preclinical, clinical and commercial manufacturing. Although technologies have been employed to further increase the percentage of bispecific antibodies in the mixtures of parental and bispecific antibodies and to decrease the percentage of miss-paired heavy and light chains, there remains a need for bispecific formats and multivalent multimer formats that eliminate or minimize some of the disadvantages mentioned above, and that increase the amount of the target moiety produced. While techniques have been previously described in the art to bolster the percentage of heterodimerization pairing of the bispecific, or multivalent multimer, there can remain unwanted impurities in the mixture of expression products. Further, separation of the heterodimerization product, which is a bispecific antibody or multivalent multimer, can be challenging, less efficient or more costly, depending on the means of separation and composition of the target moiety of interest.

Thus, there remains a need for improved and/or alternative technologies for producing and purifying biological therapeutics.

### BRIEF SUMMARY OF THE DISCLOSURE

The present invention provides novel means for producing and purifying biological therapeutics such as Ig-like molecules, bispecific antibodies and multivalent multimers. It provides novel means for improving interaction between at least two CH3 domains of interest, for example when multiple CH3 domains are present in a mixture generated by a single cell (see for example WO2013157954 and WO2013157953).

An isolated heterodimeric protein is provided herein comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, and wherein the first human IgG CH3-containing polypeptide comprises a further amino acid variant at position S364, K409 and/or K360.

Suitably, the 364 amino acid may be valine, isoleucine, threonine, glutamine or leucine.

Suitably, the 409 amino acid may be isoleucine, leucine or glutamate.

Suitably, the 360 amino acid may be aspartate.

Suitably, the heterodimeric protein may comprise a human IgG1, an IgG2, an IgG3 or an IgG4 CH3-region.

Suitably, the heterodimeric protein may comprise a human immunoglobulin Fc region. Optionally the human immunoglobulin Fc region may comprise an IgG1, an IgG2, an IgG3, or an IgG4 Fc region.

Suitably, the first CH3-containing polypeptide may be an antibody heavy chain.

Suitably, the second CH3-containing polypeptide may be an antibody heavy chain.

Suitably, the heterodimeric protein may further comprise one or more antibody light chains.

Suitably, the antibody light chain may be a common antibody light chain.

A pharmaceutical composition is also provided comprising the isolated heterodimeric protein described herein.

An isolated nucleic acid is also provided encoding the first and second human IgG CH3-containing polypeptides described herein.

A recombinant host cell is also provided comprising the isolated nucleic acid described herein.

A method of producing the isolated heterodimeric protein described herein in also provided, comprising culturing the recombinant host cell of the invention under conditions that allow expression of the first and second human IgG CH3-containing polypeptides.

A method is also provided for improving stability of a heterodimeric protein that contains a first human IgG CH3-containing polypeptide which comprises the amino acid variant L351D and L368E, and a second human IgG CH3-containing polypeptide which comprises the amino acid variant T366K and L351K, the method comprising introducing an amino acid variant into the first human IgG CH3-containing polypeptide at position S364, K409 and/or K360.

A method is also provided for decreasing the stability of a homodimeric protein that contains a first human IgG CH3-containing polypeptide which comprises the amino acid variant L351D and L368E, the method comprising introducing an amino acid variant into the first human IgG CH3-containing polypeptide at position S364, K409 and/or K360.

A method is also provided for improving the yield of a heterodimeric protein comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, comprising introducing an amino acid variant into the first human IgG CH3-containing polypeptide at position S364, K409 and/or K360.

A method is also provided for increasing the purity of a heterodimeric protein comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, comprising (a) introducing an amino acid variant into the first human IgG CH3-containing polypeptide at position S364, K409 and/or K360; and (b) subjecting the heterodimeric protein to ion exchange chromatography.

Suitably, the ion exchange chromatography may be cation exchange chromatography.

Suitably, the 364 amino acid may be valine, isoleucine, threonine, glutamine or leucine.

Suitably, the 409 amino acid may be isoleucine, leucine or glutamate.

Suitably, the 360 amino acid may be aspartate.

Suitably, the heterodimeric protein may comprise a human IgG1, an IgG2, an IgG3, or an IgG4 CH3-region.

Suitably, the heterodimeric protein may comprise a human immunoglobulin Fc region. Optionally, the human immunoglobulin Fc region may comprise an IgG1, an IgG2, an IgG3, or an IgG4 Fc region.

Suitably, the first CH3-containing polypeptide may be an antibody heavy chain.

Suitably, the second CH3-containing polypeptide may be an antibody heavy chain.

Suitably, the heterodimeric protein may further comprise one or more antibody light chains.

Suitably, the antibody light chain may be a common antibody light chain.

A heterodimeric protein produced by the method described herein is also provided.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

For the discussion that follows, where a letter is followed by a number, this indicates the amino acid at the position of the residue number in the primary amino acid sequence according to EU numbering, e.g., K351, denotes a Lysine at position 351. Where a letter is followed by a number followed by another letter, this indicates the amino acid in a native position (e.g., a wild-type human Fc) of the residue according to EU numbering, and the residue that is engineered into that position (e.g., K409S, denotes where a Lysine is present in a wild-type Fc at position 409, the engineered variation is a Serine).

Embodiments of the invention are further described hereinafter with reference to the accompanying drawings, in which:

FIG. 1: Vector map used for the expression of heavy chain variants.

FIG. 2: SDS-PAGE results for single (A) and double (B) transfections for K360 variants. Band sizes seen on non-reduced SDS-Page correspond to: 150 KDa (IgG); 75 KDa (half bodies); 50 KDa (heavy chain) and 25 KDa (light chain).

FIG. 3: SDS-PAGE results for single (A) and double (B) transfections for S364 variants. Band sizes seen on non-reduced SDS-Page correspond to: 150 KDa (IgG); 75 KDa (half bodies); 50 KDa (heavy chain) and 25 KDa (light chain).

FIG. 4: SDS-PAGE results for single and double transfections for K409 variants. Band sizes seen on non-reduced SDS-Page correspond to: 150 KDa (IgG); 75 KDa (half bodies); 50 KDa (heavy chain) and 25 KDa (light chain).

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

The invention provides methods for producing heterodimeric polypeptides, such as bispecific antibodies or multivalent multimers. It also provides for improved methods for producing heterodimeric molecules from a mixture of CH3 domain polypeptides generated from a single cell. Corresponding heterodimeric proteins, compositions, nucleic acids and host cells are also provided herein. The invention enables the generation of predominately (and in certain embodiments, almost exclusively) heterodimeric polypeptides having high yields, and mitigation of unwanted species, such as half bodies or homodimers. The methods of the invention increase heterodimer stability and reduce homodimer formation based on variations at the CH3 interface of the heterodimeric polypeptide. Details of methods and compositions are provided herein.

The terms “heterodimer”, “heterodimeric complex”, or “heterodimeric molecule” are used interchangeably herein to refer to a molecule comprising at least a first polypeptide and a second polypeptide, wherein the second polypeptide differs in amino acid sequence from the first polypeptide by at least one amino acid residue. The heterodimer can comprise a “heterodimer” formed by the first and second polypeptide or can form higher order tertiary structures where polypeptides in addition to the first and second polypeptide are present.

Except where indicated otherwise by context, the terms “first” polypeptide and “second” polypeptide, and variations thereof, are merely generic identifiers, and are not to be taken as identifying a specific or a particular polypeptide or component of heterodimers of the invention.

Various approaches are described in the art in order to promote the formation of a certain antibody of interest, thereby reducing the content of undesired antibodies or by products in the resulting mixture.

For antibodies, it is well-known that the CH3-CH3 interaction is the primary driver for Fc dimerization (Ellerson J R., et al., J. Immunol 1976 (116) 510-517; Deisenhofer J. biochemistry 1981 (20) 2361-2370). It is furthermore well-known that when two CH3 domains interact with each other they meet in a protein-protein interface which comprises “contact” residues (also called contact amino acids, interface residues or interface amino acids). Contact amino acids of a first CH3 domain interact with one or more contact amino acids of a second CH3 domain. Contact amino acids are typically within 5.5 Å (preferably within 4.5 Å) of each other in the three-dimensional structure of an antibody. The interaction between contact residues from one CH3 domain and contact residues from a different CH3 domain may for instance be via Van der Waals forces, hydrogen bonds, water-mediated hydrogen bonds, salt bridges or other electrostatic forces, attractive interactions between aromatic side chains, disulfide bonds, or other forces known to one skilled in the art. It was previously shown that approximately one-third of the contact amino acid side chains at the human IgG1 CH3 domain interface can account for the majority of contributions to domain folding and association. It can further be envisaged that other (neighbouring) amino acid residues may affect the interactions in the protein-protein interface.

Approaches to interfere with the dimerization of antibody heavy chains have been employed in the art. A number of means of engineering the CH3 domains to favour heterodimerization over homodimerization have been described in the art. An approach for the production of a given bispecific antibody of interest is based on electrostatic engineering of contact residues within the CH3-CH3 interface.

For example, an approach for the production of a given bispecific antibody or multivalent multimer of interest based on electrostatic engineering of contact residues within the CH3-CH3 interface is described in U.S. Pat. Nos. 9,248,182; 9,358,286; 9,248,182; and 9,758,805. Using a combined computational modelling and iterative experimental validation strategy to identify a novel charge-to-charge paired construct, termed “DEKK,” contact residues were identified by introducing altered charge polarity to support electrostatic interactions that favour heterodimer formation, while reducing homodimerization by charge repulsion. The L351D/T366K pair in particular, resulted in the efficient formation of heterodimers and very little homodimers. Further computational analysis was used to introduce additional electrostatic interactions at the core of the heterodimeric CH3 interface, yielding the L351D, L368E/L351K, T366K variant or DEKK, which showed high yield heterodimer formation. Furthermore, the level of aggregation, unfolding, and in vivo half-life was similar to that of natural antibodies. Structural analysis of the DEKK bispecific antibodies revealed unexpected interactions. As predicted by the computational modelling, residues Asp-351 and Glu-368 interacted with residue Lys-366 in the opposite CH3 domain. However, as a result of local shifts in the IgG backbone forming stabilizing salt-bridge interactions, residue Lys-351 did not interact with Asp-351 as predicted but formed interactions with residues Pro-352, Ser-354, and Glu-357. These interactions may account for the stability of the heterodimer molecule, making it a very attractive platform for the development of novel bispecific antibodies.

The present invention is based on heterodimeric bispecific or multivalent multimers comprising variant amino acid residues within the CH3 interface of the bispecific or multivalent multimer, which may be used in conjunction with the DEKK variant amino acid residues previously described or with other means of enhancing heterodimerization previously described in the art, such as knob-in-hole or charge engineering of the Fc (CH2 and/or CH3 domains). For example, using the above mentioned DEKK technology and including within a heavy chain comprising the DE variation further amino acid residue variations at positions K360, S364, and/or K409, it is possible to further improve the efficiency by which the heterodimeric pairings are generated compared to homodimer pairings (e.g., DEDE CH3 pairing).

Furthermore, in conditions of overexpression of one of the two heavy chain arms that comprise a desired heterodimer, this may result in production of homodimers of such arm. For example, depending on conditions, where DEKK heterodimer engineering is used, overexpression of the DE chain in relation to the amount of the KK chain, could potentially generate DEDE homodimers. Use of the variants at positions K360, S364, and/or K409 (as described herein) permit destabilization of the homodimer, generating halfbodies or single heavy chain arms (e.g., DE chains rather than DEDE homodimers), which provide for simpler and more efficient purification steps, and which when present in a mixture with a bispecific antibody or multivalent multimer cause less interference in assays testing that molecule.

Further, production of halfbodies is preferred over homodimers, as they are more readily separated out in protein batches used for research by means of gel filtration, as opposed to homodimers, which may be inseparable or more challenging to separate from heterodimers under such filtration. Furthermore, halfbodies tend to have greater separation of peaks on cation exchange chromatography (CIEX) vis-à-vis heterodimer peaks, as compared to homodimer and heterodimer peaks, making selection of a retention associated with a pure bispecific or multivalent multimer easier.

Another feature of the one or more of the amino acid variants described herein is that such variation promotes heterodimer formation, stability and/or destabilizes the homodimers, thereby increasing the overall efficiency of the expression system.

In one aspect, an invention disclosed herein provides a method for producing a heterodimeric molecule (e.g., a bispecific antibody or multivalent multimer) comprising two CH3 domains, wherein one CH3 domain comprises a CH3 variant residue(s) at positions K360, S364, and/or K409, said method comprising providing in a host cell:

a first nucleic acid encoding a 1st CH3 domain-comprising polypeptide chain, and

a second nucleic acid encoding a 2nd CH3 domain-comprising polypeptide chain,

wherein the first nucleic acid encodes one or more of the CH3 domain variant residue(s) at positions K360, S364, and/or K409; said method further comprising the step of culturing said host cell and allowing for expression of said two nucleic acids and harvesting said heterodimeric molecule from the culture.

In another aspect, an invention disclosed herein provides a method for producing a heterodimeric molecule (e.g., a bispecific antibody or multivalent multimer) comprising at least two different Ig-like molecules from a single host cell, wherein each of said two Ig-like molecules comprises two CH3 domains that are capable of forming an interface and wherein a CH3 domain of each heterodimeric molecule comprises one or more of the CH3 domain variant residues at positions K360, S364, and/or K409, said method comprising providing in a host cell:

a) a first nucleic acid encoding a 1st CH3 domain-comprising polypeptide chain,

b) a second nucleic acid encoding a 2nd CH3 domain-comprising polypeptide chain,

c) a third nucleic acid encoding a 3rd CH3 domain-comprising polypeptide chain, and

d) a fourth nucleic acid encoding a 4th CH3 domain-comprising polypeptide chain,

wherein at least two of said nucleic acids are provided with means for preferential pairing of said 1st and 2nd CH3 domain-comprising polypeptides and said 3rd and 4th CH3-domain, and wherein said nucleic acid encoding said 1st CH3 domain comprises one or more of the CH3 domain variant residues at positions K360, S364, and/or K409, and said nucleic acid encoding said 3rd CH3-domain comprises one or more of the CH3 domain variant residues at positions K360, S364, and/or K409, said method further comprising the step of culturing said host cell and allowing for expression of said at least four nucleic acid and harvesting said at least two different Ig-like molecules from the culture.

Another aspect comprises the invention above, wherein said means of preferential pairing of said 1st and 2nd CH3 domain-comprising polypeptides differs from the means of preferential pairing of said 3rd and 4th CH3-domain.

In another aspect, an invention disclosed herein provides a first nucleic acid encoding a 1st CH3 domain-comprising polypeptide chain, further comprising a DE at positions 351 and 368, respectively, and second nucleic acid encoding a 2nd CH3 domain-comprising polypeptide chain, comprising a KK at positions 351 and 366, wherein said one or more of the CH3 domain variant residues at positions K360, S364, and/or K409 are present on the 1st CH3 domain-comprising polypeptide chain, further comprising a DE at positions 351 and 368.

In another aspect, an invention disclosed herein provides a first nucleic acid encoding a 1st CH3 domain-comprising polypeptide chain, comprising variations that facilitate heterodimerization with a 2nd CH3 domain-comprising polypeptide chain encoded by a second nucleic acid comprising complementary variations that facilitate heterodimerization, wherein said first nucleic acid further encodes one or more of the CH3 domain variant residues (at positions K360, S364, and/or K409).

The phrase “cation exchange resin” refers to a solid phase that is negatively charged and has free cations for exchange with cations in an aqueous solution passed over or through the solid phase. The charge may be provided by attaching one or more charged ligands to the solid phase, e.g. by covalent linking. Alternatively, or in addition, the charge may be an inherent property of the solid phase (e.g. as is the case for silica, which has an overall negative charge).

As used herein, the term “host cell protein (HCP)” means a protein different from the recombinant cell product, such as a heterodimeric polypeptide (e.g., bispecific antibody or multivalent multimer), which is a protein derived from a source for antibody production, that is, host cells. For a bispecific antibody that may be used as a therapeutic, HCP is preferably removed from the original antibody agent. As used herein, the expression “host cell protein that is removed” is meant to include all impurities excluding the product of interest or target molecule, e.g., bispecific antibody or multivalent multimer to be purified, including, in addition to host cell protein itself, the DNA and cell growth factors derived from host cells. Thus, when the host cell protein is removed, the purity of target molecule can be increased significantly.

It is often desired to produce more than one heterodimeric protein, for instance in order to more efficiently interfere with multiple biological pathways involved in a disease process or with the invasion, replication and/or spreading of a pathogen.

A mixture of more than one heterodimeric protein is also particularly useful for the treatment of certain diseases. For example, tumor cells use many different strategies to develop resistance during treatment with antibodies or small molecule drugs. Resistance may involve multiple cell surface receptors and soluble molecules and it is considered beneficial to develop antibody-based treatments for cancers that address multiple such disease- and escape-associated mechanisms simultaneously. In case more than two such disease- and escape-related mechanisms are involved, a bispecific, multivalent multimer or mixture of heterodimeric proteins may provide an innovative and attractive therapeutic format. Preferably, such mixtures of heterodimeric proteins are produced by a single cell to facilitate a drug development process that is less complicated from a regulatory point of view and cost-effective and feasible from a drug manufacturing and clinical development point of view. In a single cell-based approach, it is desirable to use methods that allow controlled and efficient production of the bispecific antibodies, thus reducing or even completely abrogating the need of separating the desired mixture of bispecific IgG polypeptides from non-desired monospecific IgG polypeptides. In the prior art, mixtures of monospecific and bispecific antibodies have been produced by a single cell (WO2004/009618), but these mixtures represent complex concoctions of several different bispecific and monospecific antibody species. It is a further object of the present invention to provide means and methods for producing defined mixtures of heterodimer proteins (e.g., bispecific antibodies) in single cells. Preferably, methods are provided which result in mixtures of (bispecific) antibodies with a proportion of at least 95%, at least 97% or even more than 99% of dimeric IgG polypeptides, irrespective of the amount of monomeric by-products, see herein below. Typically, in a cell where multiple intact IgG polypeptides are produced, half polypeptides (monomeric by-products) may be present that can be simply removed by size exclusion chromatography known in the art.

In one embodiment, the present invention provides methods for producing a defined heterodimeric polypeptide (e.g., a bispecific antibody or multivalent multimer) or a mixture of at least two different Ig-like molecules in single cells, instead of a single (bispecific) antibody of interest, wherein the formation of other, undesired dimeric antibody species is diminished or even absent, preferably in the context of using an expression product high throughput screening, where use of CIEX purification is impractical and the presence of homodimers, as opposed to halfbodies, can significantly disrupt experimental protocols and skew experimental results. The mitigation or elimination of homodimers is accomplished by introducing variants at the CH3 interface (at positions K360, S364, and/or K409), which enhance repulsion of like CH3 domains, and may increase the attractive forces of unlike CH3 domains.

These variants can be introduced to the DE chain, in using DEKK CH3 technology. Alternatively, these variants can be introduced with other heterodimerization technology that facilitate preferential pairing of different heavy chains (heterodimer formation) over the pairing of identical heavy chains (homodimer formation), for example, such as knob-in-hole or charge engineering of the Fc (e.g., CH2 and/or CH3 domain). The resulting mixture is well defined, and its composition is controlled by the design of the CH3 domain variants.

Furthermore, regulation of expression levels and/or different transfection ratios used for expression affects the composition of the mixture. In a method according to the invention, a first nucleic acid encodes a CH3 domain which preferentially pairs with a CH3 domain encoded by a second nucleic acid, or, a first nucleic acid encodes a CH3 domain which preferentially pairs with a CH3 domain encoded by a second nucleic acid and a third nucleic acid encodes a CH3 domain which preferentially pairs with a CH3 domain encoded by a fourth nucleic acid. The present invention also provides mixtures of at least two different Ig-like molecules obtainable by the methods of the invention.

As used herein, the term “preferential pairing of said 1st and 2nd CH3 domain-comprising polypeptides” means the resulting dimers comprising the 1st CH3 domain-comprising polypeptide and/or the 2nd CH3 domain-comprising polypeptide will be dimers consisting of one 1st CH3 domain-comprising polypeptide paired with one 2nd CH3 domain-comprising polypeptide, with limited formation of dimers comprising the 1st CH3 domain-comprising polypeptide paired with another 1st CH3 domain-comprising polypeptide and 2nd CH3 domain-comprising polypeptide with another 2nd CH3 domain-comprising polypeptide. Likewise, the term “preferential pairing of said 3rd and 4th CH3 domain-comprising polypeptides” means that the resulting dimers comprising the 3rd CH3 domain-comprising polypeptide and/or the 4th CH3 domain-comprising polypeptide will be dimers consisting of one 3rd CH3 domain-comprising polypeptide paired with one 4th CH3 domain-comprising polypeptide with limited formation of dimers comprising the 3rd CH3 domain-comprising polypeptide paired with another 3rd CH3 domain-comprising polypeptide and 4th CH3 domain-comprising polypeptide with another 4th CH3 domain-comprising polypeptide. As a result, when nucleic acids encoding four different (A, B, C, D) CH3 domain-comprising polypeptides are introduced in a single cell, instead of a mixture of 10 different Ig-like dimers (AA, AB, AC, AD, BB, BC, BD, CC, CD and DD), a mixture of predominantly two bispecific Ig-like molecules is produced. The person of ordinary skill using the variants set out here will be understand that other desired combinations can be generated with this technology as well.

In a method according to the present invention, each of the above-described CH3-domain comprising polypeptide chains preferably further comprises a variable region recognizing a target epitope, or two or more variable regions. The variable regions that are part of the CH3-domain comprising polypeptide chains preferably share, and are paired with a common light chain. In that case only the VHs of the variable regions differ whereas the VL in all variable regions is essentially the same. Hence, in one aspect a method according to the invention is provided, which further comprises providing said host cell with a nucleic acid encoding a common light chain. In one embodiment, each variable region of the CH3-domain comprising polypeptide chains recognizes a different target epitope. In one embodiment, each of said 4 variable regions of the 4 CH3-domain comprising polypeptide chains recognizes a different target epitope. For instance, if the first nucleic acid encodes a heavy chain that further contains a variable domain with specificity for antigen A, the second nucleic acid encodes a heavy chain that further contains a variable domain with specificity for antigen B, the third nucleic acid encodes a heavy chain that further contains a variable domain with specificity for antigen C, and the fourth nucleic acid encodes a heavy chain that further contains a variable domain with specificity for antigen D, a mixture will then be produced containing bispecific Ig-like molecules that are specific for AB and bispecific Ig-like molecules that are specific for CD. The formation of monospecific antibodies (with AA, BB, CC or DD specificity) or bispecific antibodies with specificity for AC, AD, BC or BD is lowered or even absent due to the means for preferential pairing of said 1st and 2nd CH3 domain-comprising polypeptides and said 3rd and 4th CH3 domain-comprising polypeptides. This occurs where differing heterodimerization engineering is employed for said 1st and 2nd CH3 domain-comprising polypeptides and said 3rd and 4th CH3 domain-comprising polypeptides. It is, of course, also possible to use further nucleic acids, for instance encoding a 5th and a 6th CH3 domain-comprising polypeptide, in order to produce defined mixtures comprising more than two different Ig-like molecules (for example AB and CD).

Alternatively, said heavy chains comprising a variable domain with specificity for antigen A and B may comprise an additional variable domain with specificity for antigen C or D, connected via a linker, thereby forming a multivalent multimer, wherein heavy chains A and B may dimerize via heterodimerization engineering, such as DEKK, and homodimers are mitigated by including in one heavy chain the above variations, which destabilize homodimer formation, yielding halfbodies.

Of note, the ratio of the nucleic acids used in a method according to the invention does not need to be 1:1:1:1 and the ratio of the resulting Ig-like molecules that are expressed does not need to be 1:1. It is possible to use means known in the art to produce mixtures of antibodies with optimized ratios. For instance, expression levels of nucleic acids and hence the ratios of the resulting Ig-like molecules produced may be regulated by using different genetic elements such as promoters, enhancers and repressors or by controlling the genomic integration site of copy number of the DNA constructs encoding antibodies. Preferably, the amount of the first human IgG CH3-containing polypeptide as expressed which comprises the amino acid variant L351D and L368E and variant residues at positions S364, K409 and/or K360, is higher than the amount of the second human IgG CH3-containing polypeptide, which comprises the amino acid variant T366K and L351K. Preferably, the weight ratio of the first human IgG CH3-containing polypeptide over the second human IgG CH3-containing polypeptide, is between 1:1 and 10:1. More preferably, the amount of the DE-containing polypeptide is between 1:1 and 5:1, more preferably between 1.1 and 3:1 or between 1:1 and 2:1.

One aspect of the present invention provides a method according to the invention, wherein said means for preferential pairing of said 1st and 2nd CH3 domain-comprising polypeptides are different from said means for preferential pairing of said 3rd and 4th CH3-domain comprising polypeptides. By ‘different’ it is meant that the means for preferential pairing of said 1st and 2nd CH3 domain comprising polypeptides are designed such that preferential pairing of the 1st and 2nd chain is favoured. The design is such that essentially no interaction between the 1st and the 3rd and/or 4th CH3 domain comprising polypeptide chain will take place. In other words, dimerization between said 1st CH3 domain comprising polypeptide and said 3rd or 4th polypeptide is reduced to essentially zero and so forth. The 3rd and the 4th CH3 domain-comprising polypeptides may either be wildtype or may comprise means for preferential pairing that are different from the means for preferential pairing of the 1st and 2nd CH3 domains.

The present invention provides methods for the efficient and controlled production of a well-defined mixture of Ig like molecules, with a high proportion of bispecific antibodies in the mixture. Even a proportion of (two) bispecifics of at least 95%, at least 97% or more is obtained in a system where two bispecifics are desired. This means that only at most 5%, at most 3% or less monospecific bivalent by-products are obtained. Of note, the amount of monomeric by-products, half molecules, is less important since these half-molecules are easily separated from dimers using their size difference and, for example, cannot crosslink receptors. Accordingly, an invention described herein provides for use of CH3 variants in heavy chain arms, which upon expression of bispecific antibodies mitigates the production of homodimers in favour of halfbodies while conserving the stability of the bispecific antibody, which facilitates purification of the bispecific and mitigates homodimer interference with high throughput screening and assaying the desired bispecific antibody. Preferably, the variants of the invention provided in the CH3 domain mitigate the amount of homodimeric by-products, and enhance the formation of heterodimeric target molecules, and mitigate the amount of halfbodies produced, thereby increasing the overall efficiency of the expression system, and simplifying the downstream separation of the target molecule(s) from HCP.

In another embodiment, the variable regions of the 1st and the 2nd CH3-domain comprising polypeptide chains recognize different target epitopes, whereas the variable regions of the 3rd and the 4th CH3-domain comprising polypeptide chains recognize the same target epitopes. This will result in the predominant production of one kind of bispecific Ig-like molecule and one kind of monospecific Ig-like molecule. For instance, if the variable regions of the 1st and the 2nd CH3-domain comprising polypeptide chains recognize different target epitopes and if the variable regions of the 3rd and the 4th CH3-domain comprising polypeptide chains both recognize the same target epitope which is different from the target epitopes recognized by the 1st and the 2nd CH3-domains, a mixture of Ig-like molecules having specificity for AB or CC will be formed. Further provided is therefore a method according to the invention, wherein the target epitope recognized by the variable regions of the 3rd and 4th CH3 domain comprising polypeptide chain is the same, but different from the target epitope recognized by the variable region of the 1st or the 2nd CH3-domain comprising polypeptide chain. Alternatively, when the variable regions of the 1st and the 2nd CH3-domain comprising polypeptide chains recognize different target epitopes and when the variable regions of the 3rd and the 4th CH3-domain comprising polypeptide chains both recognize the same epitope as the 1st or the 2nd CH3-domain comprising polypeptide chains, a mixture of Ig-like molecules having specificity for AB and AA, or AB and BB will be formed. A method according to the invention, wherein the target epitope recognized by the variable regions of the 3rd and 4th CH3 domain comprising polypeptide chain is the same as the target epitope recognized by the variable region of the 1st or the 2nd CH3-domain comprising polypeptide chain is therefore also herewith provided.

In some embodiments, the heterodimeric polypeptide of the invention is an immunoglobulin-like (Ig-like) polypeptide. The term ‘Ig-like polypeptide’ as used herein means a proteinaceous moiety that possesses at least one immunoglobulin (Ig) domain. Said Ig-like polypeptide comprises a sequence comprising the function of at least an immunoglobulin CH3 domain, preferably the sequence comprises an IgG1 CH3 domain. Proteinaceous moieties that possess at least a CH3 domain can be further equipped with specific binding moieties. The CH3 domains of the present invention, containing means for preferential pairing, can thus be used for preferential pairing of two CH3-domain comprising proteinaceous moieties to design desired heterodimeric binding molecules or mixtures of binding molecules. Binding moieties that can be engineered to the CH3-domain comprising proteinaceous moieties can be any binding agent, including, but not limited to, single chain Fvs, single chain or Tandem diabodies (TandAb®), VHHs, Anticalins®, Nanobodies®, a BiTE®, a Fab, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins® or a KALBITOR®. In one embodiment, the binding moieties are antibody variable regions (VH/VL combinations). Variable regions that are part of the CH3-domain comprising polypeptide chains preferably share a common light chain. In that case, only the VHs of the variable regions differ whereas the VL in all variable regions is essentially the same.

Alternatively, or in addition, other polypeptides can be engineered to the CH3 domains of the present invention, including cytokines, hormones, soluble ligands, receptors and/or peptides.

In one embodiment, said Ig-like polypeptide comprises a full length Fc backbone. In another embodiment, the Ig-like polypeptides are antibodies. The variable regions of these antibodies preferably share a common light chain, but they may differ in their VH regions. The term ‘antibody’ as used herein means a proteinaceous molecule belonging to the immunoglobulin class of proteins, containing one or more domains that bind an epitope on an antigen, where such domains are derived from or share sequence homology with the variable region of an antibody. Antibodies are known in the art and include several isotypes, such as IgG1, IgG2, IgG3, IgG4, IgA, IgD, IgE, and IgM. An antibody according to the invention may be any of these isotypes, or a functional derivative and/or fragment of these. In one embodiment, Ig-like molecules are produced that are antibodies of the IgG, IgA, IgD, IgE, or IgE isotype. In one embodiment, Ig like molecules are produced that are antibodies of the IgG isotype because IgG antibodies e.g. have a longer half-life as compared to antibodies of other isotypes.

Antibodies produced with methods according to the present invention can have sequences of any origin, including murine, avine, such as chicken, and human sequences. Antibodies can consist of sequences from one origin only, such as fully human antibodies, or they can have sequences of more than one origin, resulting for instance in chimeric or humanized antibodies. Antibody binding can be expressed in terms of specificity and affinity. The specificity determines which antigen or epitope thereof is bound by the binding domain. The affinity is a measure for the strength of binding to a particular antigen or epitope. The term ‘antigen’ as used herein means a substance or molecule that may be bound by the antigen-binding site of an antibody. Also, mixtures of antigens can be regarded as ‘antigen’; the skilled person would appreciate that a lysate of tumor cells, or viral particles may be indicated as ‘antigen’ whereas such tumor cell lysate or viral particle preparation exists of many antigenic determinants. An antigen comprises at least one, but often more, epitopes. The term ‘epitope’ as used herein means a part of an antigen that is recognized by the immune system, specifically by antibodies, B cells, or T cells.

The term ‘CH3 domain’ is well known in the art. The IgG structure has four chains, two light and two heavy chains; each light chain has two domains, the variable and the constant light chain (VL and CL) and each heavy chain has four domains, the variable heavy chain (VH) and three constant heavy chain domains (CH1, CH2, CH3). The CH2 and CH3 domain region of the heavy chain is called Fc (Fragment crystallizable) portion, Fc fragment, Fc backbone or simply Fc. The IgG molecule is a heterotetramer having two heavy chains that are held together by disulfide bonds (—S—S—) at the hinge region and two light chains attached to the heavy chains via —S—S— disulphide bonds. The heavy chains dimerize including through interactions at the CH3-CH3 domain interface and through interactions at the hinge region. The number of hinge disulfide bonds varies among the immunoglobulin subclasses (Papadea and Check 1989). The Fc fragment of an immunoglobulin molecule is a dimer of the two C-terminal constant regions, CH2 and CH3 domains, of the heavy chain. Among its physiological functions are interactions with the complement system and with specific receptors on the surface of a variety of cells. Interactions between the CH3 domains of two individual heavy chains are known to play an important role in driving heavy chain dimerization. Thus, CH3 domains direct the association of antibody heavy chains (Deisenhofer J., Biochemistry 1981(20)2361-2370; Miller S., J. Mol. Biol. 1990(216)965-973; Padlan, Advances in Protein Chemistry 1996 (49) 57-133). The CH3 variants of the present invention can thus be used in association with other antibody domains to generate full length antibodies that are either bispecific or monospecific. The specificity of the antibody as defined by the VH/VL combinations typically does not affect the heavy chain dimerization behaviour that is driven by the CH3 domains.

Contact residues within the CH3-CH3 interface can either be amino acids that are charged, or amino acid residues that are neutral at physiological conditions. The term ‘charged amino acid residue’ or ‘charged residue’ as used herein means amino acid residues with electrically charged side chains. These can either be positively charged side chains, such as may be present in arginine (Arg, R), histidine (His, H) and lysine (Lys, K) or can be negatively charged side chains, such as may be present in aspartic acid (Asp, D) and glutamic acid (Glu, E). The term ‘neutral amino acid residue’ or neutral residue as used herein refers to all other amino acids that do not carry electrically charged side chains. These neutral residues include serine (Ser, S), threonine (Thr, T), asparagine (Asn, N), glutamine (Glu, Q), Cysteine (Cys, C), glycine (Gly, G), proline (Pro, P), alanine (Ala, A), valine (Val, V), isoleucine (Ile, I), leucine (Leu, L), methionine (Met, M), phenylalanine (Phe, F), tyrosine (Tyr, Y), and tryptophan (Trp, T).

The term ‘CH3-CH3 domain interface’, or ‘CH3 interface’, ‘CH3-CH3 pairing’, ‘domain interface’ or simply ‘interface’, as used herein, refers to the association between two CH3 domains of separate CH3-domain comprising polypeptides that is a result of interacting amino acid residues, comprising at least one interaction between an amino acid of a first CH3 domain and an amino acid of a second CH3 domain. Such interaction is for instance via Van der Waals forces, hydrogen bonds, water-mediated hydrogen bonds, salt bridges or other electrostatic forces, attractive interactions between aromatic side chains, the formation of disulfide bonds, or other forces known to one skilled in the art.

The present invention further provides a method for making a host cell for production of at least two different Ig-like polypeptides, the method comprising the step of introducing into said host cell nucleic acid sequences encoding at least a first and a second CH3-domain comprising polypeptide chain, wherein at least two of said nucleic acid sequences are provided with means for preferential pairing of said first and second CH3-domain comprising polypeptides, wherein said nucleic acid sequences are introduced consecutively or concomitantly. Likewise, a host cell may already have introduced into said host cell nucleic acid sequences encoding a least a first and a second CH3 domain comprising polypeptide chain, wherein nucleic acid encoding variable regions(s) are integrated adjacent to the CH3 domain encoding nucleic acid, such that the host cell can be used as a master cell for integration of different variable regions for the production of different heterodimeric molecules, wherein the at least two of said nucleic acid sequences encoding at least a first and a second CH3 domain provide a means for preferential pairing of said first and second CH3-domain comprising polypeptides.

It is a further aspect of the present invention to provide a method for making a host cell for production of a heterodimeric polypeptides, the method comprising the step of introducing into said host cell nucleic acid sequences encoding at least a first and a second CH3-domain comprising polypeptide chain, wherein said first CH3 domain-comprising polypeptide chain comprises a positively charged amino acid residue where a wildtype CH3 typically has a neutral amino acid residue at that position and wherein said second CH3 domain-comprising polypeptide chain comprises a negatively charged amino acid residue where a wildtype CH3 typically has a neutral amino acid residue at that position, wherein said nucleic acid sequences are introduced consecutively or concomitantly. Said methods for making said host cells preferably further comprise the step of introducing into said host cell a nucleic acid sequence encoding a common light chain, or alternatively, said methods integrate said nucleic acids sequences into a host cell that already has a nucleic acid sequence encoding a common light chain transiently, but preferably stably integrated into the host cell.

Also provided herein is a recombinant host cell comprising nucleic acid sequences encoding at least a first, a second, a third and a fourth CH3-domain comprising polypeptide chain, wherein at least two of said nucleic acids are provided with means for preferential pairing of said first and second CH3-domain comprising polypeptides and said third and fourth CH3-domain comprising polypeptides.

The invention furthermore provides a recombinant host cell comprising nucleic acid sequences encoding at least a first and a second CH3-domain comprising polypeptide chain, wherein said first CH3 domain-comprising polypeptide chain comprises a positively charged amino acid residue where a wildtype CH3 typically has a neutral amino acid residue at that position and wherein said second CH3 domain-comprising polypeptide chain comprises a negatively charged amino acid residue where a wildtype CH3 typically has a neutral amino acid residue at that position.

A recombinant host cell according to the invention can further comprise a nucleic acid encoding a common light chain.

A “host cell” according to the invention may be any host cell capable of expressing recombinant DNA molecules, including bacteria such as for instance Escherichia (e.g. E. coli), Enterobacter, Salmonella, Bacillus, Pseudomonas, Streptomyces, yeasts such as S. cerevisiae, K. lactis, P. pastoris, Candida, or Yarrowia, filamentous fungi such as Neurospora, Aspergillus oryzae, Aspergillus nidulans and Aspergillus niger, insect cells such as Spodoptera frugiperda SF-9 or SF-21 cells, and preferably mammalian cells such as Chinese hamster ovary (CHO) cells, BHK cells, mouse cells including SP2/0 cells and NS-0 myeloma cells, primate cells such as COS and Vero cells, MDCK cells, BRL 3A cells, hybridomas, tumor-cells, immortalized primary cells, human cells such as W138, HepG2, HeLa, HEK293, HT1080 or embryonic retina cells such as PER. C6, and the like. Often, the expression system of choice will involve a mammalian cell expression vector and host so that the antibodies can be appropriately glycosylated. A human cell line can be used to obtain antibodies with a completely human glycosylation pattern. The conditions for growing or multiplying cells (see e. g. Tissue Culture, Academic Press, Kruse and Paterson, editors (1973)) and the conditions for expression of the recombinant product may differ somewhat, and optimization of the process is usually performed to increase the product proportions and/or growth of the cells with respect to each other, according to methods generally known to the person skilled in the art. In general, principles, protocols, and practical techniques for maximizing the productivity of mammalian cell cultures can be found in Mammalian Cell Biotechnology: a Practical Approach (M. Butler, ed., IRL Press, 1991). Expression of antibodies in recombinant host cells has been extensively described in the art. The nucleic acids encoding the light and heavy chains may be present as extrachromosomal copies and/or stably integrated into the chromosome of the host cell.

It is a further aspect of the present invention to provide a culture of recombinant host cells according to the invention, or a culture of recombinant host cells obtainable or obtained by a method according to the invention, said culture either producing one or more heterodimeric polypeptides.

To obtain expression of nucleic acid sequences encoding the CH3 domain-comprising polypeptides, it is well known to those skilled in the art that sequences capable of driving such expression can be functionally linked to the nucleic acid sequences encoding the CH3 domain-comprising polypeptides. Functionally linked is meant to describe that the nucleic acid sequences encoding the CH3 domain-comprising polypeptides or precursors thereof is linked to the sequences capable of driving expression such that these sequences can drive expression of the CH3 domain-comprising polypeptides or precursors thereof. Useful expression vectors are available in the art, e.g. the pcDNA vector series of Invitrogen. Where the sequence encoding the polypeptide of interest is properly integrated with reference to sequences governing the transcription and translation of the encoded polypeptide, the resulting expression cassette is useful to produce the polypeptide of interest, referred to as expression. Sequences driving expression may include promoters, enhancers and the like, and combinations thereof. These should be capable of functioning in the host cell, thereby driving expression of the nucleic acid sequences that are functionally linked to them. Promoters can be constitutive or regulated, and can be obtained from various sources, including viruses, prokaryotic, or eukaryotic sources, or artificially designed. Expression of nucleic acids of interest may be from the natural promoter or derivative thereof or from an entirely heterologous promoter. Some well-known and much used promoters for expression in eukaryotic cells comprise promoters derived from viruses, such as adenovirus, e.g. the E1A promoter, promoters derived from cytomegalovirus (CMV), such as the CMV immediate early (IE) promoter, promoters derived from Simian Virus 40 (SV40), and the like. Suitable promoters can also be derived from eukaryotic cells, such as methallothionein (MT) promoters, elongation factor 1a (EF-1a) promoter, actin promoter, an immunoglobulin promoter, heat shock promoters, and the like. Any promoter or enhancer/promoter capable of driving expression of the sequence of interest in the host cell is suitable in the invention. In one embodiment the sequence capable of driving expression comprises a region from a CMV promoter, preferably the region comprising nucleotides −735 to +95 of the CMV immediate early gene enhancer/promoter. The skilled artisan will be aware that the expression sequences used in the invention may suitably be combined with elements that can stabilize or enhance expression, such as insulators, matrix attachment regions, STAR elements (WO 03/004704), and the like. This may enhance the stability and/or levels of expression. Protein production in recombinant host cells has been extensively described, e.g. in Current Protocols in Protein Science, 1995, Coligan J E, Dunn B M, Ploegh H L, Speicher D W, Wingfield P T, ISBN 0-471-11184-8; Bendig, 1988. Culturing a cell is done to permit it to metabolize, and/or grow and/or divide and/or produce recombinant proteins of interest. This can be accomplished by methods well known to persons skilled in the art, and includes but is not limited to providing nutrients for the cell. The methods comprise growth adhering to surfaces, growth in suspension, or combinations thereof. Several culturing conditions can be optimized by methods well known in the art to optimize protein production yields. Culturing can be done for instance in dishes, flasks, roller bottles or in bioreactors, using batch, fed-batch, continuous systems, hollow fiber, and the like. In order to achieve large scale (continuous) production of recombinant proteins through cell culture, cells capable of growing in suspension are employed, and the cells are capable of being cultured in the absence of animal- or human-derived serum or animal- or human-derived serum components. Thus, purification is easier and safety is enhanced due to the absence of additional animal or human proteins derived from the culture medium, while the system is also very reliable as synthetic media are the best in reproducibility.

Immunoglobulin-like polypeptides are expressed in host cells and are harvested from the cells or, preferably, from the cell culture medium by methods that are generally known to the person skilled in the art. After harvesting, these Ig-like polypeptides may be separated by using methods known in the art. Such methods may include precipitation, centrifugation, filtration, size-exclusion chromatography, affinity chromatography, cation- and/or anion-exchange chromatography, hydrophobic interaction chromatography, and the like. For a mixture of antibodies comprising IgG polypeptides, protein A or protein G affinity chromatography can be suitably used (see e.g. U.S. Pat. Nos. 4,801,687 and 5,151,504).

Following capture using affinity chromatography, orthogonal polishing steps are used to remove any remaining process-related impurities, which may include homodimers, charge variants, HCP, and DNA. In general, to obtain a separated bispecific antibody or multivalent multimer, the following steps are undertake, including host cell culture, harvest clarification, followed by protein capture, anion exchange chromatography, including to remove host cell DNA, then CIEX is used to remove host cell protein, leached protein A and potential aggregates followed by additional steps, such as virus filtration. Persons of skill in the art are aware the order of such steps may be modified, or individual steps substituted or eliminated. For example, alternatives for the second polishing step include hydrophobic interaction chromatography and mixed-mode chromatography.

Immunoglobulin-like polypeptides, and/or mixtures thereof, produced with methods according to the present invention preferably have a common light chain. Further provided is, therefore, a method according to the invention, further comprising providing said host cell with a nucleic acid encoding a common light chain. This is a light chain that is capable of pairing with at least two different heavy chains, thereby forming functional antigen binding domains. A functional antigen binding domain is capable of specifically binding to an antigen. In one embodiment, a common light chain is used that is capable of pairing with all heavy chains produced with a method according to the invention, thereby forming functional antigen binding domains, so that mispairing of unmatched heavy and light chains is avoided. In one aspect, only common light chains with one identical amino acid sequence are used. Alternatively, those of skill in the art will recognize that “common” also refers to functional equivalents of the light chain of which the amino acid sequence is not identical. Many variants of said light chain exist wherein mutations (deletions, substitutions, additions) are present that do not materially influence the formation of functional binding regions. Such variants are thus also capable of binding different heavy chains and forming functional antigen binding domains. The term ‘common light chain’ as used herein thus refers to light chains which may be identical or have some amino acid sequence differences while retaining the binding specificity of the resulting antibody after pairing with a heavy chain. It is for instance possible to prepare or find light chains that are not identical but still functionally equivalent, e.g. by introducing and testing conservative amino acid changes, and/or changes of amino acids in regions that do not or only partly contribute to binding specificity when paired with the heavy chain, and the like. A combination of a certain common light chain and such functionally equivalent variants is encompassed within the term “common light chain”. Reference is made to WO 2004/009618 for a detailed description of the use of common light chains. Preferably, a common light chain is used in the present invention which is a germline-like light chain, more preferably a germline light chain, preferably a rearranged germline human kappa light chain, most preferably either the rearranged germline human kappa light chain IgVκ1-39/Jκ or IGVκ3-20/Jκ. A common light chain preferably comprises a light chain variable region as noted above with 0-5 amino acid insertions, deletions, substitutions, additions or a combination thereof. Another preferred common light chain is the human kappa light chain IgVκ1-39/IGJκ5. Preferably, an antibody of the invention comprises the variable region of human kappa light chain IgVκ1-39/IGJκ5. A further preferred common light chain is the human kappa light chain IgVκ3-15/IGJκ1. Preferably, an antibody of the invention comprises the variable region of human kappa light chain IgVκ3-15/IGJκ1. A further preferred common light chain is the human kappa light chain IgVκ3-20/IGJκ1. Preferably, an antibody of the invention comprises the variable region of human kappa light chain IgVκ3-20/IGJκ1. A further preferred common light chain is the human lambda light chain IgVλ3-21/IGJλ3. Preferably, an antibody of the invention comprises the variable region of human kappa light chain IgVλ3-21/IGJλ3.

Alternatively, the skilled person may select, as an alternative to using a common light chain and to avoid mispairing of unmatched heavy and light chains, means for forced pairing of the heavy and light chain, such as for example described in WO2009/080251, WO2009/080252 and/or WO2009/080253.

The inventors have previously described expression of CH3 amino acids having charged residues where wildtype CH3 has a neutral amino acid residue at that position, such that the alternative charges of two CH3 domains cause a stable interaction (e.g., a first CH3 having positively charged residues at positions 351 and 366 and a second CH3 having negatively charged residues at positions 351 and 368). The variations and modified heavy chains of the present invention may be incorporated with the above means of promoting heterodimerization or other means of doing so known to persons of skill in the art. The inventions disclosed herein provide further capacity to promote heterodimerization of CH3 domains, and may also increase stability of the heterodimerization or decrease the stability of the homodimer based on amino acid residue variant(s) at the CH3 interface, and, preferably a combination of the foregoing benefits.

The heterodimers according to the invention are more stable as compared to the wild type dimers (the wild type dimer is defined as a bispecific IgG (AB) without CH3 engineering in contrast to its homodimers (AA or BB)), and more readily separable from mixtures and impurities. It has surprisingly become possible to increase the proportion of one or more Ig-like polypeptides of interest in a mixture even further. Methods known in the art for preferential production of a heterodimer (e.g., bispecific antibody) typically involves the production of some undesired dimeric side products. For instance, the proportion of a bispecific antibody of interest using a knob-into-hole technology known to those of skill the art is at best reportedly 87%, whereas the electrostatic engineering approach wherein charged contact amino acids are substituted by amino acids of opposite charge, also results in proportions reportedly of up to 96%. The present inventors have succeeded in introducing variations that further enhance the proportion of a heterodimeric polypeptide of interest.

The present invention is directed to CH3 domain(s) comprising at least one variant residue at position K360, S364, and/or K409. These variant residues are described in more detail below. Where a letter is followed by a number followed by another letter, this indicates the amino acid in a native position (e.g., a wild-type human Fc) of the residue according to EU numbering, and the residue that is engineered into that position (e.g., K409S, denotes where a Lysine is present in a wild-type Fc at position 409, the engineered variation is a Serine). For EU numbering see for example: http://www.imgt.org/IMGTScientificChart/Numbering/HuIGHGnber.html.

One embodiment of the present invention provides a method for producing a heterodimeric polypeptide from a single cell, wherein said heterodimeric polypeptide comprises amino acid variants at positions K360, S364, and/or K409, said method comprising providing in said cell a. A first nucleic acid encoding a 1st CH3 domain-comprising polypeptide chain, b. A second nucleic acid encoding a 2nd CH3 domain-comprising polypeptide chain, wherein the 1st CH3 domain-comprising polypeptide comprise variants at positions K360, S364, and/or K409, said method further comprising the step of culturing said host cell and allowing for expression of said two nucleic acids and harvesting said heterodimeric Ig-like polypeptides from the culture.

One embodiment of the present invention therefore provides a heterodimeric polypeptide, wherein said heterodimeric polypeptide comprises two CH3 domains, wherein said 1st CH3 domain-comprising polypeptide chain comprises the DE amino acid variants, and wherein said 2nd CH3 domain-comprising polypeptide chain comprises the KK amino acid variants and, wherein one or more residues at one or more positions K360, S364 and/or K409 in said 1st CH3 domain-comprising polypeptide chain is a variant as compared to the human wild-type residue.

One embodiment of the present invention provides a method or producing a heterodimeric polypeptide from a single cell, comprising providing in said cell

a. A first nucleic acid encoding a 1st CH3 domain-comprising polypeptide chain,

b. A second nucleic acid encoding a 2nd CH3 domain-comprising polypeptide chain,

wherein the 1st CH3 domain-comprising polypeptide chains comprise the DE and further amino acid variants at positions K360, S364, and/or K409,

wherein the 2nd CH3 domain-comprising polypeptide chains comprise the KK, and said method further comprising the step of culturing said host cell and allowing for expression of said two nucleic acids and harvesting said heterodimeric Ig-like polypeptides from the culture.

In another embodiment the variant residue(s) at positions K360, S364, and/or K409 is combined with other heterodimerization variations, comprising, for example, knob-into-hole, opposite charge substitutions at the CH3 region or other techniques known to persons of ordinary skill in the art.

In one embodiment, the method further comprises the step of providing said host cell with a nucleic acid encoding a common light chain, which has advantages as outlined herein before.

As stated elsewhere herein, a heterodimeric protein is described comprising a first CH3-containing polypeptide and a second CH3-containing polypeptide, wherein said first CH3-containing polypeptide comprises a further amino acid variant at position S364, K409 and/or K360. Optionally, the first and second CH3 polypeptides are human.

For example, the first CH3-containing polypeptide described herein may comprise a S364V amino acid.

Alternatively, the first CH3-containing polypeptide described herein may comprise a S3641 amino acid.

In another example, the first CH3-containing polypeptide described herein may comprise a S364T amino acid.

In a further example, the first CH3-containing polypeptide described herein may comprise a S364Q amino acid.

Alternatively, the first CH3-containing polypeptide described herein may comprise a S364L amino acid.

The first CH3-containing polypeptide described herein may comprise an amino acid variant at position K409 as an alternative or in addition to an amino acid a variant at position S364 (e.g. S364V, S3641, S364T, S364Q or S364L as described in detail above).

Accordingly, the first CH3-containing polypeptide described herein may comprise a K409I amino acid, optionally in addition to an amino acid a variant at position S364 (e.g. S364V, S3641, S364T, S364Q or S364L as described in detail above).

Alternatively, the first CH3-containing polypeptide described herein may comprise a K409L amino acid, optionally in addition to an amino acid a variant at position S364 (e.g. S364V, S3641, S364T, S364Q or S364L as described in detail above).

In a further example, the first CH3-containing polypeptide described herein may comprise a K409E amino acid, optionally in addition to an amino acid a variant at position S364 (e.g. S364V, S3641, S364T, S364Q or S364L as described in detail above).

The first CH3-containing polypeptide described herein may comprise an amino acid variant at position K360 as an alternative or in addition to an amino acid a variant at position S364 (e.g. S364V, S3641, S364T, S364Q or S364L as described in detail above) and/or a variant at position K409 (e.g. K409I, K409L or K409E as described in detail above).

Accordingly, the first CH3-containing polypeptide described herein may comprise a K360D amino acid, optionally in addition to an amino acid a variant at position S364 (e.g. S364V, S3641, S364T, S364Q or S364L as described in detail above) and/or optionally in addition to an amino acid variant at position K409 (e.g. K409I, K409L or K409E as described in detail above).

In one example, the heterodimeric protein comprises a first human IgG CH3-containing polypeptide and a second human IgG CH3-containing polypeptide, wherein the first human IgG CH3-containing polypeptide comprises a further amino acid variant at position S364, K409 and/or K360. Optionally, said first human IgG CH3-containing polypeptide further comprises the amino acid variant L351D and L368E and said second human IgG CH3-containing polypeptide comprises the amino acid variant T366K and L351K.

For example, the first human IgG CH3-containing polypeptide described herein may comprise a S364V amino acid.

Alternatively, the first human IgG CH3-containing polypeptide described herein may comprise a S3641 amino acid.

In another example, the first human IgG CH3-containing polypeptide described herein may comprise a S364T amino acid.

In a further example, the first human IgG CH3-containing polypeptide described herein may comprise a S364Q amino acid.

Alternatively, the first human IgG CH3-containing polypeptide described herein may comprise a S364L amino acid.

The first human IgG CH3-containing polypeptide described herein may comprise an amino acid variant at position K409 as an alternative or in addition to an amino acid a variant at position S364 (e.g. S364V, S3641, S364T, S364Q or S364L as described in detail above).

Accordingly, the first human IgG CH3-containing polypeptide described herein may comprise a K409I amino acid, optionally in addition to an amino acid a variant at position S364 (e.g. S364V, S3641, S364T, S364Q or S364L as described in detail above).

Alternatively, the first human IgG CH3-containing polypeptide described herein may comprise a K409L amino acid, optionally in addition to an amino acid a variant at position S364 (e.g. S364V, S3641, S364T, S364Q or S364L as described in detail above).

In a further example, the first human IgG CH3-containing polypeptide described herein may comprise a K409E amino acid, optionally in addition to an amino acid a variant at position S364 (e.g. S364V, S3641, S364T, S364Q or S364L as described in detail above).

The first human IgG CH3-containing polypeptide described herein may comprise an amino acid variant at position K360 as an alternative or in addition to an amino acid a variant at position S364 (e.g. S364V, S3641, S364T, S364Q or S364L as described in detail above) and/or a variant at position K409 (e.g. K409I, K409L or K409E as described in detail above).

Accordingly, the first human IgG CH3-containing polypeptide described herein may comprise a K360D amino acid, optionally in addition to an amino acid a variant at position S364 (e.g. S364V, S3641, S364T, S364Q or S364L as described in detail above) and/or optionally in addition to an amino acid variant at position K409 (e.g. K409I, K409L or K409E as described in detail above).

In one example, a heterodimeric protein is described comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, and wherein the first human IgG CH3-containing polypeptide comprises a further amino acid variant at position S364, K409 and/or K360.

Accordingly, in one embodiment, a heterodimeric protein is provided comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, and wherein the first human IgG CH3-containing polypeptide comprises a further amino acid variant at position S364.

In a particular example, the 364 amino acid is valine, isoleucine, threonine, glutamine or leucine.

In one embodiment, the 364 amino acid is valine.

In one embodiment, the 364 amino acid is isoleucine.

In one embodiment, the 364 amino acid is threonine.

In one embodiment, the 364 amino acid is glutamine.

In one embodiment, the 364 amino acid is leucine.

In another embodiment, a heterodimeric protein is provided comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, and wherein the first human IgG CH3-containing polypeptide comprises a further amino acid variant at position K409.

In a particular example, the 409 amino acid is isoleucine, leucine or glutamate.

In one embodiment, the 409 amino acid is isoleucine.

In one embodiment, the 409 amino acid is leucine.

In one embodiment, the 409 amino acid is glutamate.

In another embodiment, a heterodimeric protein is provided comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, and wherein the first human IgG CH3-containing polypeptide comprises a further amino acid variant at position K360.

In a particular example, the 360 amino acid is aspartate.

The amino acid variants described herein may refer to a single variation at position S364, position K409, or position K360 within the “DE” arm of the heterodimeric proteins of the invention. In additional embodiments of the invention, the heterodimeric proteins have at least two variations at positions selected from: S364, K409 or K360. In further embodiments of the invention, the heterodimeric proteins have at least three further variations at positions S364, K409 and K360.

Therefore, in one embodiment a heterodimeric protein is provided comprising a first human IgG CH3-containing polypeptide and a second human IgG CH3-containing polypeptide, wherein said first human IgG CH3-containing polypeptide comprises the amino acid variant L351 D and L368E and said second human IgG CH3-containing polypeptide comprises the amino acid variant T366K and L351K, and wherein the first human IgG CH3-containing polypeptide comprises a further amino acid variant at position S364 and K409, wherein the 364 amino acid is valine, isoleucine, threonine, glutamine or leucine and the 409 amino acid is isoleucine, leucine or glutamate.

For example, the first human IgG CH3-containing polypeptide described herein may comprise a S364V amino acid and a K409I amino acid. Alternatively, the first human IgG CH3-containing polypeptide described herein may comprise a S364V amino acid and a K409L amino acid. In one example, the first human IgG CH3-containing polypeptide described herein may comprise a S364V amino acid and a K409E amino acid.

In another example, the first human IgG CH3-containing polypeptide described herein may comprise a S3641 amino acid and a K409I amino acid. Alternatively, the first human IgG CH3-containing polypeptide described herein may comprise a S3641 amino acid and a K409L amino acid. In one example, the first human IgG CH3-containing polypeptide described herein may comprise a S3641 amino acid and a K409E amino acid.

In another example, the first human IgG CH3-containing polypeptide described herein may comprise a S364T amino acid and a K409I amino acid. Alternatively, the first human IgG CH3-containing polypeptide described herein may comprise a S364T amino acid and a K409L amino acid. In one example, the first human IgG CH3-containing polypeptide described herein may comprise a S364T amino acid and a K409E amino acid.

In another example, the first human IgG CH3-containing polypeptide described herein may comprise a S364Q amino acid and a K409I amino acid. Alternatively, the first human IgG CH3-containing polypeptide described herein may comprise a S364Q amino acid and a K409L amino acid. In one example, the first human IgG CH3-containing polypeptide described herein may comprise a S364Q amino acid and a K409E amino acid.

In another example, the first human IgG CH3-containing polypeptide described herein may comprise a S364L amino acid and a K409I amino acid. Alternatively, the first human IgG CH3-containing polypeptide described herein may comprise a S364L amino acid and a K409L amino acid. In one example, the first human IgG CH3-containing polypeptide described herein may comprise a S364L amino acid and a K409E amino acid.

In another embodiment a heterodimeric protein is provided comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, and wherein the first human IgG CH3-containing polypeptide comprises a further amino acid variant at position S364 and K360, wherein the 364 amino acid is valine, isoleucine, threonine, glutamine or leucine and the 360 amino acid is aspartate.

For example, the first human IgG CH3-containing polypeptide described herein may comprise a S364V amino acid and a K360D amino acid.

In another example, the first human IgG CH3-containing polypeptide described herein may comprise a S3641 amino acid and a K360D amino acid.

In another example, the first human IgG CH3-containing polypeptide described herein may comprise a S364T amino acid and a K360D amino acid.

In another example, the first human IgG CH3-containing polypeptide described herein may comprise a S364Q amino acid and a K360D amino acid.

In another example, the first human IgG CH3-containing polypeptide described herein may comprise a S364L amino acid and a K360D amino acid.

In another embodiment a heterodimeric protein is provided comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, and wherein the first human IgG CH3-containing polypeptide comprises a further amino acid variant at position K409 and K360, wherein the 409 amino acid is isoleucine, leucine or glutamate and the 360 amino acid is aspartate.

For example, the first human IgG CH3-containing polypeptide described herein may comprise a K409I amino acid and a K360D amino acid.

In another example, the first human IgG CH3-containing polypeptide described herein may comprise a K409L amino acid and a K360D amino acid.

In another example, the first human IgG CH3-containing polypeptide described herein may comprise a K409E amino acid and a K360D amino acid.

In another embodiment a heterodimeric protein is provided comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, and wherein the first human IgG CH3-containing polypeptide comprises a further amino acid variant at position S364, K409 and K360, wherein the 364 amino acid is valine, isoleucine, threonine, glutamine or leucine; the 409 amino acid is isoleucine, leucine or glutamate; and the 360 amino acid is aspartate.

For example, the first human IgG CH3-containing polypeptide described herein may comprise S364V, K409I and K360D. Alternatively, the first human IgG CH3-containing polypeptide described herein may comprise S364V, K409L and K360D. In one example, the first human IgG CH3-containing polypeptide described herein may comprise S364V, K409E and K360D.

In another example, the first human IgG CH3-containing polypeptide described herein may comprise S3641, K409I and K360D. Alternatively, the first human IgG CH3-containing polypeptide described herein may comprise S3641, K409L and K360D. In one example, the first human IgG CH3-containing polypeptide described herein may comprise S3641, K409E and K360D.

In another example, the first human IgG CH3-containing polypeptide described herein may comprise S364T, K409I and K360D. Alternatively, the first human IgG CH3-containing polypeptide described herein may comprise S364T, K409L and K360D. In one example, the first human IgG CH3-containing polypeptide described herein may comprise S364T, K409E and K360D.

In another example, the first human IgG CH3-containing polypeptide described herein may comprise S364Q, K409I and K360D. Alternatively, the first human IgG CH3-containing polypeptide described herein may comprise S364Q, K409L and K360D. In one example, the first human IgG CH3-containing polypeptide described herein may comprise S364Q, K409E and K360D.

In another example, the first human IgG CH3-containing polypeptide described herein may comprise S364L, K409I and K360D. Alternatively, the first human IgG CH3-containing polypeptide described herein may comprise S364L, K409L and K360D. In one example, the first human IgG CH3-containing polypeptide described herein may comprise S364L, K409E and K360D.

In a particular embodiment, the 360 amino acid is aspartate and the 364 amino acid is isoleucine.

In another embodiment, the 360 amino acid is aspartate and the 364 amino acid is threonine.

In a further embodiment, the 360 amino acid is aspartate and the 364 amino acid is valine.

In yet another embodiment, the 360 amino acid is aspartate and the 409 amino acid is glutamate.

In a further embodiment, the 364 amino acid is leucine and the 409 amino acid is leucine.

In yet another embodiment, the 364 amino acid is threonine and the 409 amino acid is glutamate.

Variant polypeptides may be created by any suitable means known in the art. Variants described herein may be based on a human IgG CH3 domain, but then with one or more parent amino acids (at positions 360, 364 and/or 409) varied with the corresponding position of the human IgG CH3 amino acid described herein.

The sequences of human IgG CH3 domains are well known in the art. By varying one or more amino acids in the polypeptide, the side chains at those positions are altered. The amino acid variations described herein may be introduced into nucleic acids encoding a CH3 domain or into the CH3 domain itself through a variation of means, including genetic means using molecule biology, or via enzymatic or chemical means. Appropriate methods for generating the variants described herein are well known in the art. For example, variant nucleic acids may be generated de novo via nucleic acid synthesis that encode the variant sequences and ordered through any number of providers. Similarly, variant nucleic acids can be generated through mutagenesis techniques, well known in the art. Such nucleic acids encoding the variant polypeptide that may then be cloned into host cells, expressed and assayed, if desired. These practices are carried out using well-known procedures, and a variety of methods that may find use in are described in Molecular Cloning—A Laboratory Manual, 3rd Ed. (Maniatis, Cold Spring Harbor Laboratory Press, New York, 2001), and Current Protocols in Molecular Biology (John Wiley & Sons), both entirely incorporated by reference. The nucleic acids that encode the variants may be incorporated into an expression vector in order to express the protein. Expression vectors typically include a promoter linked with control or regulatory sequences, selectable markers, any fusion partners, and/or additional elements. The variants may be produced by culturing a host cell transformed with nucleic acid, preferably an expression vector, containing nucleic acid encoding the variants, under the appropriate conditions to induce or cause expression of the protein. A wide variety of appropriate host cells may be used, including but not limited to mammalian cells, bacteria, insect cells, and yeast. For example, a variety of cell lines that may find use are described in the ATCC cell line catalog, available from the American Type Culture Collection, entirely incorporated by reference. The methods of introducing exogenous nucleic acid into host cells are well known in the art, and will vary with the host cell used.

In one aspect of the invention, a method according to the invention for producing at least two different Ig-like molecules or for producing a heterodimeric Ig-like molecule is provided wherein each of the CH3-domain comprising polypeptide chains further comprises a variable region recognizing a different target epitope, wherein the target epitopes are located on the same molecule. This often allows for more efficient counteraction of the (biological) function of said target molecule as compared to a situation wherein only one epitope is targeted. For example, a heterodimeric immunoglobulin-like molecule may simultaneously bind to 2 epitopes present on, e.g., growth factor receptors or soluble molecules critical for tumors cells to proliferate, thereby effectively blocking several independent signalling pathways leading to uncontrolled proliferation, and any combination of at least two Ig-like molecules may simultaneously bind to 2, or even 3 or 4 epitopes present on such growth factor receptors or soluble molecules.

In one embodiment, the target molecule is a soluble molecule. In another embodiment, the target molecule is a membrane-bound molecule.

In another aspect of the invention, a method according to the invention for producing one or more heterodimeric immunoglobulin-like molecules is provided wherein each of the CH3-domain comprising polypeptide chains further comprises a variable region recognizing a target epitope, wherein the target epitopes are located on different molecules. In this case, each of the different target molecules may either be a soluble molecule or a membrane-bound molecule. In one embodiment, the different target molecules are soluble molecules. Alternatively, one target molecule is a soluble molecule whereas the second target molecule is a membrane bound molecule. In yet another alternative, both target molecules are membrane bound molecules. In one embodiment the different target molecules are expressed on the same cells, whereas in other embodiments the different target molecules are expressed on different cells. As a non-limiting example, any heterodimeric immunoglobulin-like molecule or any combination of at least two immunoglobulin-like molecules may be suitable for simultaneously blocking multiple membrane-bound receptors, neutralizing multiple soluble molecules such as cytokines or growth factors for tumor cells or for neutralizing different viral serotypes or viral strains.

One embodiment provides a method according to the invention for producing one or more heterodimeric immunoglobulin-like molecule, wherein at least one of said target epitopes is located on a tumor cell. Alternatively, or additionally, at least one of said target epitopes is located on the surface of an effector cell. This is for instance suitable for recruitment of T cells or NK cells for tumor cell killing. For instance, at least one Ig-like molecule is produced with a method according to the invention that is capable of recruiting immune effector cells, preferably human immune effector cells, by specifically binding to a target molecule located on immune effector cells. In a further embodiment, said immune effector cell is activated upon binding of the immunoglobulin-like molecule to the target molecule. Recruitment of effector mechanisms may for instance encompass the redirection of immune modulated cytotoxicity by administering an immunoglobulin-like molecule produced by a method according to the invention that is capable of binding to a cytotoxic trigger molecule such as the T cell receptor or an Fc gamma receptor, thereby activating downstream immune effector pathways. The term ‘immune effector cell’ or ‘effector cell’ as used herein refers to a cell within the natural repertoire of cells in the mammalian immune system which can be activated to affect the viability of a target cell. Immune effector cells include cells of the lymphoid lineage such as natural killer (NK) cells, T cells including cytotoxic T cells, or B cells, but also cells of the myeloid lineage can be regarded as immune effector cells, such as monocytes or macrophages, dendritic cells and neutrophilic granulocytes. Hence, said effector cell is preferably an NK cell, a T cell, a B cell, a monocyte, a macrophage, a dendritic cell or a neutrophilic granulocyte.

In one aspect the present invention thus provides methods according to the invention for producing a heterodimeric immunoglobulin-like polypeptide, wherein each of the CH3-domain comprising polypeptide chains further comprises a variable region recognizing a target epitope. In one embodiment, each of the 2 variable regions of the CH3-domain comprising polypeptide chains recognizes the same target epitope but with different affinities. In another embodiment, each of the 2 variable regions of the CH3-domain comprising polypeptide chains recognizes a different target epitope. In another embodiment, the different target epitopes are located on the same target molecule, which can be either a membrane-bound molecule or a soluble molecule. In another embodiment, the different target epitopes are located on different target molecules, which can be either expressed on the same cells or on different cells. Alternatively, the different target molecules can be soluble molecules, or one target molecule can be a soluble molecule whereas the second target molecule is a membrane bound molecule. In one embodiment, at least one of the target molecules of the heterodimeric immunoglobulin-like polypeptide is located on a tumor cell. In yet another embodiment, at least one of the target molecules of the heterodimeric Ig-like polypeptide is located on an effector cell.

In one embodiment, a method according to the invention for producing one or more heterodimeric immunoglobulin-like polypeptides is provided, wherein said polypeptides are antibodies. In one embodiment, the antibodies are of the IgG, IgA, IgD, IgE, or IgM isotype. In one embodiment, the antibodies are of the IgG isotype. In another embodiment, the antibodies are of the IgG1 isotype.

Further provided is a heterodimeric immunoglobulin-like polypeptide, or a mixture of at least two Ig-like polypeptides, obtainable by a method according to the present invention. Said heterodimeric polypeptide or mixture of polypeptides comprises two CH3 domains, wherein one of said two CH3 domains comprises the amino acid variants L351D and L368E and wherein the other of said two CH3 domains comprises the amino acid variants T366K and L351K, including one or more variant to the wild-type CH3 sequence that enhances heterodimerization and/or destabilizes homodimer formation, and preferably accomplishes a combination of these benefits, comprising variation at positions 360, 364, and 409 according to EU numbering.

These amino acid variants cause preferential pairing of said two CH3 domains, as explained before. The amino acid variants L351D and L368E in one of said two CH3 domains and the amino acid variants T366K and L351K in the other of said two CH3 domains are together dubbed DEKK herein. The CH3 domain that carries the amino acid variants L351D and L368E is also dubbed ‘the DE-side’, “the DE arm”, or “the DE” and the CH3 domain that carries the amino acid variants T366K and L351K is also dubbed ‘the KK-side’, “the KK arm”, or “the KK”. The amino acid variations described herein (at positions K360, S364, and/or K409) may be introduced alone or preferably with other variations to promote heterodimerization, and more preferably on the DEKK backbone, and more preferably on the DE-side. Whereas the variants at amino acid positions 360, 364, and 409 are capable of bolstering stability of the heterodimer and/or destabilizing the homodimer.

Also provided is a pharmaceutical composition comprising a heterodimeric immunoglobulin-like polypeptide, or a mixture of at least two polypeptides obtainable by any method according to the invention. In one embodiment, the heterodimeric polypeptides is/are antibody/antibodies or multivalent multimers. Said pharmaceutical composition may comprise heterodimeric immunoglobulin-like polypeptide, or a mixture of at least two polypeptides comprising monospecific or bispecific Ig-like molecules, or a combination of monospecific and bispecific Ig-like polypeptides. In addition, a pharmaceutical composition according to the invention comprises a pharmaceutically acceptable carrier. As used herein, such ‘pharmaceutically acceptable carrier’ includes solvents, salts, dispersion media, coatings, antibacterial and antifungal agents, isotonic and absorption delaying agents, and the like that are physiologically compatible. Depending on the route of administration (e.g., intravenously, subcutaneously, intra-articularly and the like) the Ig-like polypeptides may be coated in a material to protect the Ig-like polypeptides from the action of acids and other natural conditions that may inactivate the Ig-like polypeptides.

In one embodiment the invention provides a heterodimeric antibody comprising a first heavy chain with a first human IgG CH3-region and a second heavy chain with a second human IgG CH3-region that is different from the first human IgG CH3-region, wherein the first human CH3-region comprises the IgG CH3 amino acid variant L351D and L368E and the second human CH3-region comprises the human IgG CH3 amino acid variant T366K and L351K, and wherein the first human IgG CH3-region comprises a further amino acid variant at positions K360, S364, or K409. The heterodimeric antibody preferably comprises one or more antibody light chains. The first and second human IgG CH3-regions are preferably human IgG1, IgG2, IgG3 or IgG4 regions. The first and second human IgG CH3-regions are preferably IgG1 CH3 regions. The first heavy chain may comprise a heavy chain variable region that is different from a heavy chain variable region of the second heavy chain. Antibody variable domains comprising the different heavy chain variable regions associated with an appropriate light chain may bind different epitopes and/or antigens. The first heavy chain and/or the second heavy chain may comprise more than one variable region. A heterodimeric antibody comprising one or two heavy chain with more than one variable region can form three or more variable domains together with the appropriate complementary variable regions. Heterodimeric antibodies with two or more variable domains that bind different epitopes and/or antigens are also referred to as bi-, tri, tetra, etc specific antibodies. Such antibodies are also collectively referred to as multispecific antibodies.

For the purpose of clarity and a concise description features are described herein as part of the same or separate embodiments, however, it will be appreciated that the scope of the invention may include embodiments having combinations of all or some of the features described.

The invention is further illustrated by the following examples. These examples are not limiting the invention in any way, but merely serve to clarify the invention.

Unless defined otherwise herein, all technical and scientific terms used herein have the same meaning as commonly understood by one of ordinary skill in the art to which this invention pertains. Also, as used herein, the singular terms “a”, “an” include the plural reference. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Example 1: In Silico Predictions

A human IgG Fc variant comprising DEKK (L351D, L368E/L′351K, T′366K) was used as a starting model. WO2013/157954; De Nardis et al, J. Biol. Chem. (2017) 292(35) 14706-14717. Structural studies were performed in silico to identify key contact residues in the CH3 DEKK/DEDE interface. These residues were analyzed using High Ambiguity Driven protein-protein DOCKing (HADDOCK; an information-driven docking approach that models the docking of biomolecular complexes, e.g. proteins or nucleic acids) to identify variants that substantially reduce the formation of DEDE homodimers, while not significantly negatively impacting or improving the stability of the DEKK interface. A total of 18 human IgG CH3 variants were selected for in vitro analysis, all in the DEKK background. Each of the 18 variants had a different variant amino acid at one of the following positions in the human IgG DE CH3 domain: K360, S354, S364 or K409.

### Example 2: In Vitro SDS-PAGE Experiments

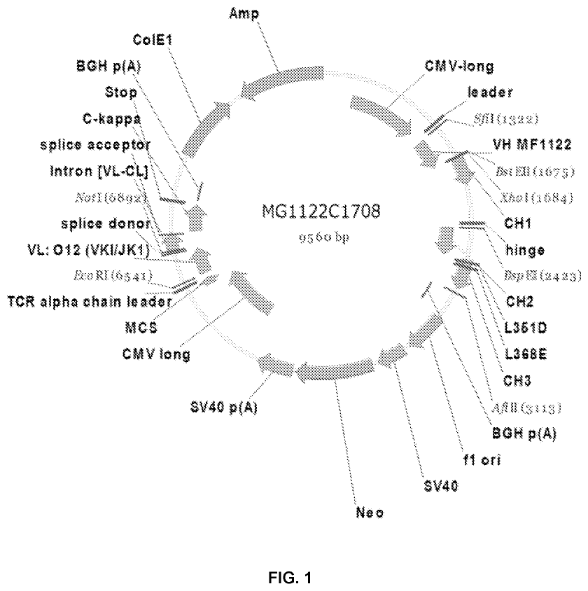

IgG vectors with all proposed variants in CH3 in addition to the L351D, L368E (DE) mutations were generated. For this purpose, 18 constructs were generated. Productions were performed using the single DE* heavy chain as well as combined transfections of DE* and KK heavy chains, where * denotes one of the 18 variations noted above. The constructs were cloned into MG1122C1708 (FIG. 1) using BspEI and AfIII restriction enzymes. Colony/sequence PCR was performed on the ligation products in order to retrieve all variants. All confirmed variants were miniprepped, followed by sequencing to confirm CH2, CH3 and VH identity.

Transfections of single DE* constructs (labelled PGxxxx IgG) and DE* constructs combined with KK constructs bearing a different VH (labelled PBxxxxx bispecific IgG) into 293FF cells were performed. After a 7 days of incubation (37° C., 8% CO₂, 155 rpm) cell supernatant was harvested, followed by protein A purification (acidic elution) and buffer exchange to PBS pH 7.4. Consequently, the IgG concentration of the samples was measured by Octet.

SDS-PAGE Analysis

All produced and purified IgGs were analyzed with non-reducing SDS-PAGE. For each production plate, the same amount of IgG sample was loaded in each well, typically 1 μg was loaded (if this was not possible, the amount was adjusted to the sample with the lowest concentration). This allowed direct comparisons to be made between the samples. The gels were examined by eye for their full IgG and halfbody content. A Chemidoc MP Imager software can also be used to quantify the relative amounts of full IgG and halfbody in each gel.

Larger amounts of halfbodies in single (PGxxxx) transfections indicated that the variant homodimer is unstable. IgG bands in the double transfections (PBxxxxx) confirmed that a heterodimer is formed.

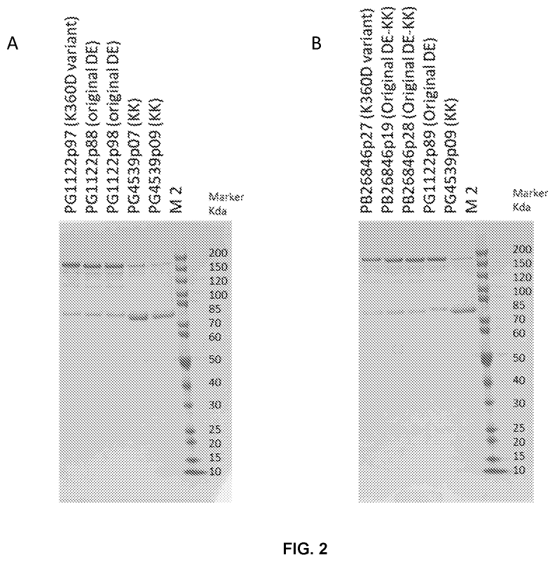

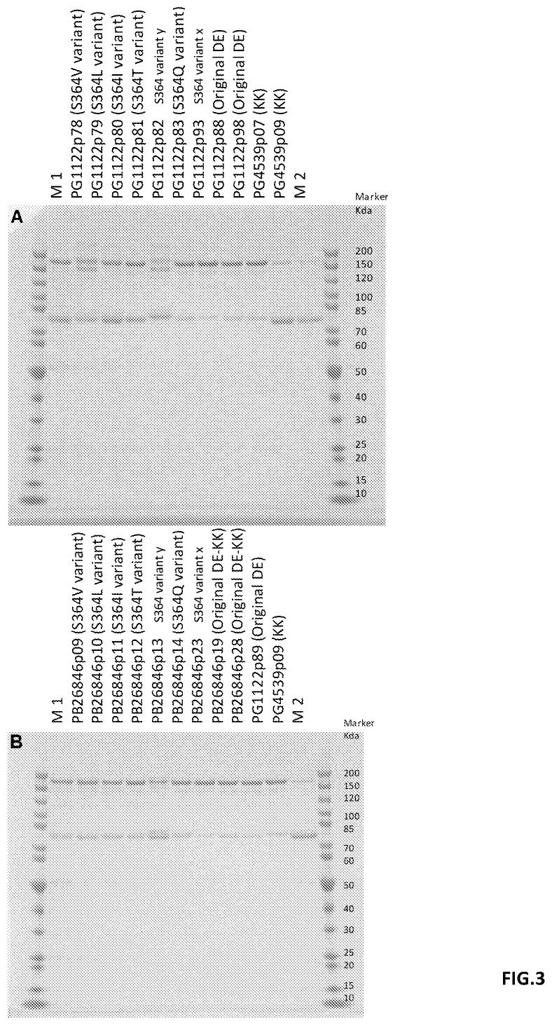

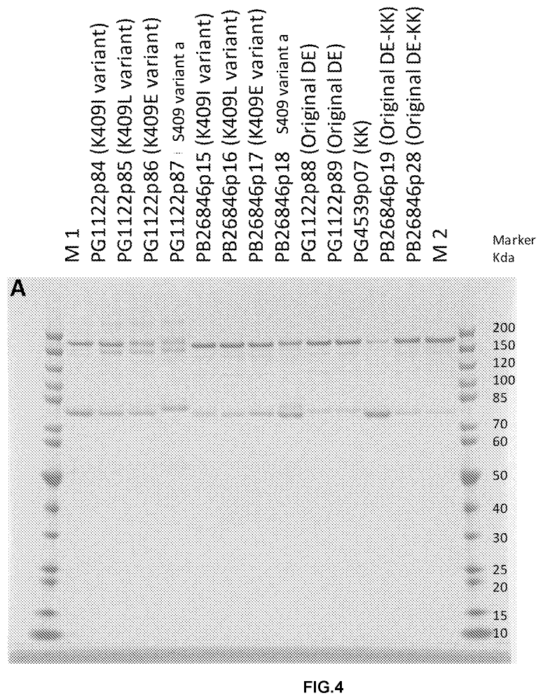

DE*-CH3 variants that showed mostly halfbody formation in the single transfections (comparable to the MG4539C1452 control sample containing the KK CH3 region) and formed mostly full IgG in the bispecific transfections were selected for further characterization in a follow-up study (FIGS. 2 to 4 and Table 1). A total of three of the four positions identified in silico had improved properties in vitro as compared to the control samples (K360, S364, and K409). A total of 9 variants were selected for further study.

TABLE 1
Summary of the results observed in SDS-PAGE experiments. All are in the context
of a CH3 that contains DE.

| | | Selected for |
|---|---|---|
| Variant | | further |
| name | comment | analysis? |
| K360D | Bispecifics; less halfbodies seen on SDS-page | Yes |
| | (less loaded 0.6 μg compared to 0.7 ug for control) | |
| S364V | more halfbodies in single production compared to original DE | Yes |
| S364I | more halfbodies in single production compared to original DE | Yes |
| S364T | more halfbodies in single production compared to original DE | Yes |
| S364Q | possibly more halfbodies in single production compared to | Yes |
| | original DE | |
| K409I | more halfbodies in single production compared to original DE | Yes |
| S364L | Extra bands seen around 140/160/>200 Kda on SDS-page | Yes |
| | (single production) | |
| K409L | Extra bands seen by single production on SDS-page | Yes |
| K409E | Extra bands seen by single and bispecific production on SDS-page, | Yes |
| | a bit more half bodies seen by double transfection | |

Following the SDS-PAGE experiments, further in silico HADDOCK analysis was performed to identify potentially promising double variants (Table 2, row 10 to 15) that could be further improved as compared to the controls.

TABLE 2
Summary of mutants selected for further study

| | | Experimental results |
|---|---|---|
| # | DEKK | Reference |
| 1 | K360D | Less halfbodies in bispecific production |
| 2 | S364I | more halfbodies in single production |
| 3 | S364Q | possibly more halfbodies in single |
| | | production |
| 4 | S364T | more halfbodies in single production |
| 5 | S364V | more halfbodies in single production |
| 6 | S364L | Additional bands in single production |
| 7 | K409I | more halfbodies in single production |
| 8 | K409L | Additional bands in single production |
| 9 | K409E | Additional bands in single production |
| 10 | S364I/K360D | Follow-up experiments described below |
| 11 | S364T/K360D | Follow-up experiments described below |
| 12 | S364T/K409E | Follow-up experiments described below |
| 13 | K360D/K409E | Follow-up experiments described below |
| 14 | S364V/K360D | Follow-up experiments described below |
| 15 | S364L/K409L | Follow-up experiments described below |

In summary a total of 18 variants were made at 3 positions in the DE arm of CH3. Single transfections were carried out to obtain homodimers, and double transfection with a common KK arm was carried out to obtain bispecific IgG. SDS-PAGE analysis showed that 5 DEKK variants showed more halfbody contaminants in single-arm productions than the DEKK control sample. A further 3 variants were selected despite aberrant SDS-PAGE results in single-production, because of the results for bispecific IgG in the double transfection experiments. Combinations of well-performing mutations were analyzed using HADDOCK, from which 6 additional combinations were selected for further analysis.

### Example 3: In Vitro Stability Results and Biophysical Characterization

The 15 selected variants were produced as bispecifics (CD137 (MF6744—previously published in US Pub. No. 2020/0017595 A1; US Pub. No. 2019/00352401 A2.) x Fibrinogen (MF1122)) with DE:KK ratios of 1:1 and 3:1, wherein the DE-arm was overexpressed to study its homodimer formation.

The IgGs were characterized further using CIEX-HPLC (cation exchange-high performance liquid chromatography), HP-SEC (high performance size exclusion chromatography) and nMS (native Mass Spectrometry) techniques. Additionally, a stability analysis was performed analyzing melting and aggregation temperatures Tm and Tagg. The variants that significantly reduced homodimer formation and demonstrated good Fc stability as indicated by high Tm/Tagg were selected for larger scale protein productions.

The VH as used differ in weight (weight monoclonal IgG MF1122 144905 and for MF6744 145932 kDa) and therefore homodimers and heterodimers can be separated on nMS. They differ in CIEX RT (13.3 min and 9.7 min) and therefore homo- and heterodimers can be separated in CIEX, especially since they will be combined with KK and DE respectively. The Fab have a high Tm. High-Tm Fab allowed the melting temperatures (Tm) of the variant CH3s to be identified.

The generated IgGs with DNA ratio 1:1 were analyzed in a Tm/Tagg analysis using the UNcle instrument (UNchained Labs Product Code 200-1037). The melting temperature (Tm) is determined by following the change of intrinsic fluorescence of amino acids upon temperature increase (spectral range 250-720 nM). The aggregation temperature (Tagg) is determined by measuring Static Light Scattering (SLS) simultaneously to the determination of Tm, using lasers with wavelengths of 266 nm and 473 nm and detection of the scattered light at an angle of 90°. DLS (dynamic light scattering) analysis was performed by Fluorescence and SLS acquisition (Blue&UV, exposure time 1000 ms, UV266: filter 4, Blue473: Filter 3) using a linear temperature ramp from 25° C.-95° C. with a rate of 0.3° C./min for Tm/Tagg determination.

This allowed determination of the Tm/Tagg of non-stressed samples with a KK:DE ratio of 1:1. All samples were at 300 μg/mL, as shown in Table 4.

Stress Tests

A small stability assay was performed by stressing the modified IgG, these assays were all performed in PBS.

Three 100 μL aliquots of each sample was generated at 300 μg/mL (as shown in Table 3).

The following stresses were applied to the samples:
5×FT (freeze thawing) (samples were repeatedly stored at −80 C for at least 1 h, followed by complete thawing at room temperature);
2 days at 50° C. (samples were placed into a water bath or incubator at 50° C. and incubated for 2 days);
Storage at 4° C. (as a non-stressed control).

The stressed samples were subsequently analyzed with DLS in direct comparison to non-stressed samples (kept at 4° C.) to determine the presence of aggregate in the sample.

Whilst some differences in the Tm/Tagg of the variants was observed they were all considered stable molecules (Tables 3 and 4).

TABLE 3
Summary of DLS results,

| | | | 50° C. | | | | | F/T | | |
|---|---|---|---|---|---|---|---|---|---|---|
| | | | | Pk 1 | | Pk 2 | | | Pk 1 | |
| | | | | Mode | Pk 1 | Mode | Pk2 | | Mode | Pk 1 |
| | | | T | Dia | Mass | Dia | Mass | T | Dia | Mass |
| Well | Sample | Variant | (° C.) | (nm) | (%) | (nm) | (%) | (° C.) | (nm) | (%) |
| A1 | 0.3 mg/ml PB31852p33 | DE + S364V | 24.95 | 10.72 | 100 | — | | 24.97 | 9.53 | 100 |
| B1 | 0.3 mg/ml PB31852p34 | DE + S364L | 25.05 | 10.75 | 100 | — | | 24.97 | 10.33 | 100 |
| C1 | 0.3 mg/ml PB31852p35 | DE + S364I | 25 | 11.2 | 100 | — | | 24.95 | 10.32 | 100 |
| D1 | 0.3 mg/ml PB31852p36 | DE + S364T | 24.97 | 12.13 | 99.81 | 60.39 | 0.19 | 24.96 | 10.33 | 100 |
| E1 | 0.3 mg/ml PB31852p37 | DE + K409I | 25.04 | 10.35 | 100 | — | | 24.95 | 10.72 | 100 |
| F1 | 0.3 mg/ml PB31852p38 | DE + K409L | 24.97 | 10.33 | 100 | — | | 24.99 | 10.34 | 100 |
| G1 | 0.3 mg/ml PB31852p39 | DE + K409E | 25.01 | 11.64 | 100 | — | | 25.07 | 10.75 | 100 |
| H1 | 0.3 mg/ml PB31852p40 | DE + S354Q | 24.97 | 14.24 | 100 | — | | 25.02 | 9.91 | 100 |
| I1 | 0.3 mg/ml PB31852p47 | DE + S364T_K409E | 24.99 | 11.2 | 100 | — | | 24.98 | 9.54 | 100 |
| J1 | 0.3 mg/ml PB31852p42 | DE + K360D_S364l | 24.97 | 11.19 | 100 | — | | 25 | 9.9 | 100 |
| K1 | 0.3 mg/ml PB31852p43 | DE + K360D_S364T | 24.95 | 10.32 | 99.92 | 76.8 | | 24.97 | 10.33 | 100 |
| L1 | 0.3 mg/ml PB31852p44 | DE + K360D_S364V | 24.99 | 10.73 | 100 | — | | 24.99 | 9.9 | 100 |
| M1 | 0.3 mg/ml PB31852p45 | DE + K360D_K409E | 25.07 | 11.66 | 100 | — | | 25 | 9.54 | 100 |
| N1 | 0.3 mg/ml PB31852p46 | DE + S364L_K409L | 24.97 | 10.72 | 100 | — | | 25.01 | 10.34 | 100 |
| A2 | 0.3 mg/ml PB31852p47 | DE + S364T_K409E | 24.98 | 10.33 | 100 | — | | 25 | 9.9 | 100 |
| B2 | 0.3 mg/ml PB31852p48 | DE only | 24.96 | 11.19 | 100 | — | | 24.99 | 10.34 | 100 |

| | | | F/T | | 4° C | | | | |
|---|---|---|---|---|---|---|---|---|---|
| | | | Pk 2 | | | Pk 1 | | Pk 2 | |
| | | | Mode | Pk 2 | | Mode | Pk 1 | Mode | Pk 2 |
| | | | Dia | Mass | T | Dia | Mass | Dia | Mass |
| Well | Sample | Variant | (nm) | (%) | (° C.) | (nm) | (%) | (nm) | (%) |
| A1 | 0.3 mg/ml PB31852p33 | DE + S364V | | | 25 | 9.9 | 100 | — | |
| B1 | 0.3 mg/ml PB31852p34 | DE + S364L | | | 24.96 | 9.89 | 100 | — | |
| C1 | 0.3 mg/ml PB31852p35 | DE + S364I | | | 24.97 | 9.89 | 99.98 | 94.83 | |
| D1 | 0.3 mg/ml PB31852p36 | DE + S364T | | | 25.02 | 9.91 | 99.97 | 94.95 | |
| E1 | 0.3 mg/ml PB31852p37 | DE + K409I | | | 24.98 | 9.54 | 99.97 | 83.28 | |
| F1 | 0.3 mg/ml PB31852p38 | DE + K409L | | | 25 | 10.34 | 100 | — | |
| G1 | 0.3 mg/ml PB31852p39 | DE + K409E | | | 24.99 | 10.34 | 99.93 | 60.42 | |
| H1 | 0.3 mg/ml PB31852p40 | DE + S354Q | | | 25 | 9.9 | 100 | — | |
| I1 | 0.3 mg/ml PB31852p47 | DE + S364T_K409E | | | 25 | 9.54 | 99.97 | 76.89 | |
| J1 | 0.3 mg/ml PB31852p42 | DE + K360D_S364l | | | 24.99 | 9.54 | 100 | — | |
| K1 | 0.3 mg/ml PB31852p43 | DE + K360D_S364T | | | 25 | 10.34 | 100 | — | |
| L1 | 0.3 mg/ml PB31852p44 | DE + K360D_S364V | | | 24.99 | 9.54 | 100 | — | |
| M1 | 0.3 mg/ml PB31852p45 | DE + K360D_K409E | | | 25 | 10.34 | 100 | — | |
| N1 | 0.3 mg/ml PB31852p46 | DE + S364L_K409L | | | 24.99 | 10.34 | 100 | — | |
| A2 | 0.3 mg/ml PB31852p47 | DE + S364T_K409E | | | 24.98 | 10.33 | 100 | — | |
| B2 | 0.3 mg/ml PB31852p48 | DE only | | | 24.99 | 10.34 | 100 | — | |

Pk1 = peak 1, pk2 = peak 2, Dia = diameter.

TABLE 4
Summary of Tm results generated using the UNcle instrument.
Note that all samples tested here have the PBcode PB31852 as a
prefix to their unique identifier number (e.g. p33, p34 etc).
This nomenclature is also abbreviated to the unique identifier numbers (e.g.
PB31852p33 is abbreviated to p33) within the examples section, e.g.
in Table 5 for simplicity. Details of the variant amino acids
corresponding to each unique identifier can be found,
for example, in Table 3.

| | | | | | Tagg |
|---|---|---|---|---|---|
| | | Tm1 | Tm2 | Tm3 | 266 |
| Well | Sample | (° C.) | (° C.) | (° C.) | (° C.) |
| A1 | 0.3 mg/ml | 64 | 74.9 | | 75.19 |
| | PB31852p33_4C | | | | |
| B1 | 0.3 mg/ml | 63 | 74.9 | | 75.15 |
| | PB31852p34_4C | | | | |
| C1 | 0.3 mg/ml | 64.11 | 75 | 83.06 | 74.56 |
| | PB31852p35_4C | | | | |
| D1 | 0.3 mg/ml | 54.17 | 65.7 | 75.06 | 74.64 |
| | PB31852p36_4C | | | | |
| E1 | 0.3 mg/ml | 65 | 75.07 | | 74.74 |
| | PB31852p37_4C | | | | |
| F1 | 0.3 mg/ml | 66.03 | 75 | | 74.8 |
| | PB31852p38_4C | | | | |
| G1 | 0.3 mg/ml | 62.5 | 75.14 | | 74.84 |
| | PB31852p39_4C | | | | |
| H1 | 0.3 mg/ml | 54.56 | 64.5 | 74.59 | 74.65 |
| | PB31852p40_4C | | | | |
| I1 | 0.3 mg/ml | 62.5 | 75 | 93.04 | 75.13 |
| | PB31852p47_4C | | | | |
| J1 | 0.3 mg/ml | 64.67 | 75.16 | | 74.82 |
| | PB31852p42_4C | | | | |
| K1 | 0.3 mg/ml | 65.5 | 75.18 | 90 | 74.86 |
| | PB31852p43_4C | | | | |
| L1 | 0.3 mg/ml | 64.24 | 75.5 | 83.5 | 75.51 |
| | PB31852p44_4C | | | | |
| M1 | 0.3 mg/ml | 62.5 | 76 | 85.12 | 75.95 |
| | PB31852p45_4C | | | | |
| N1 | 0.3 mg/ml | 65.08 | 76 | 84.5 | 75.66 |
| | PB31852p46_4C | | | | |
| A2 | 0.3 mg/ml | 64 | 77.5 | | 77.55 |
| | PB31852p47_4C | | | | |
| B2 | 0.3 mg/ml | 67.66 | 77.5 | | 76.75 |
| | PB31852p48_4C | | | | |
| | (DE only) | | | | |
| O2 | 0.25 mg/ml | 72 | 80.54 | | 81.59 |
| | PG1122p113 | | | | |

HP-SEC

The generated IgG were analyzed on HP-SEC according to standard methods known in the art. Agilent 1260 series HPLC system, Tosoh TSK-gel G3000SWxl cat #808541 with Tosoh TSK guard column SWXL cat #808543, running buffer: 200 mM NaPO₄, 50 mM NaCl, pH 7.0, 20 μg of sample loaded. HP-SEC analysis was performed to detect smaller aggregates as compared to the DLS analysis (Table 5) and was performed after 1×F/T cycle to represent samples normally used in experiments after having been stored at −80° C.

TABLE 5
Summary of HP-SEC results (wherein p48 and p64
are control DE arms), main peak represents biclonics
and/or homodimer IgG, late-eluting peak
contains halfbodies.

| | | Main peak | | Late eluting | |
|---|---|---|---|---|---|
| | | area % of | | peak % of | |
| 1:1 | 1:3 | total1:1 | Main 3:1 | total 1:1 | Late 3:1 |
| p33 | p49 | 74.82 | 57.65 | 25.18 | 41.63 |
| p34 | p50 | 86.75 | 77.41 | 12.19 | 20.6 |
| p35 | p51 | 79.38 | 60 | 20.62 | 38.87 |
| p36 | p52 | 82.32 | 68.92 | 17.68 | 30.36 |
| p37 | p53 | 75.51 | 54.53 | 22.18 | 44.85 |
| p38 | p54 | 92.7 | 83.82 | 5.84 | 14.94 |
| p39 | p55 | 88.31 | 81.81 | 11.16 | 16.58 |
| p40 | p56 | 91.68 | 90.54 | 7.12 | 7.66 |
| p47 | p63 | 79.21 | 59.09 | 20.28 | 40.26 |
| p42 | p58 | 77.77 | 54.51 | 21.48 | 44.37 |
| p43 | p59 | 80.59 | 67.75 | 17.21 | 31.01 |
| p44 | p60 | 73.86 | 55.59 | 24.57 | 44.11 |
| p45 | p61 | 88.9 | 85.64 | 9.07 | 13.39 |
| p46 | p62 | 94.4 | 89.18 | 4.88 | 9.96 |
| p47 | p63 | 80.98 | 57.93 | 18.6 | 40.85 |
| p48 | p64 | 94.08 | 87.76 | 4.42 | 11.02 |

The stress-test shows that the DEKK variants are quite stable, and only show aggregation after severe stress.

TABLE 6
Summary of HP-SEC results after the samples were kept at 4° C.

| | | 4° C. | | | | |
|---|---|---|---|---|---|---|
| | | | | | | Late |
| | | Early Early (%) | Early (%) | Main (%) | Late (%) | Late (%) |
| 1:1 | Variant | Aggregates | Aggregates | IgG | Halfbody | Fragments |
| p33 | DE + S364V | | 0.55 | 74.61 | 24.84 | |
| p34 | DE + S364L | | 0.53 | 86.81 | 12.66 | |
| p35 | DE + S364I | 0.89 | 0.60 | 78.09 | 20.42 | |
| p36 | DE + S364T | 0.76 | 0.72 | 81.05 | 17.46 | |
| p37 | DE + K409I | 0.82 | 0.92 | 76.42 | 21.84 | |
| p38 | DE + K409L | 0.24 | 0.57 | 93.23 | 5.96 | |
| p39 | DE + K409E | 0.78 | 0.66 | 86.74 | 11.83 | |
| p40 | DE + S354Q | 0.32 | 1.02 | 91.50 | 7.16 | |
| p47 | DE + S364T_K409E | | 0.52 | 79.14 | 20.34 | |
| p42 | DE + K360D_S364I | | 0.35 | 78.38 | 21.26 | |
| p43 | DE + K360D_S364T | 0.28 | 0.63 | 81.25 | 17.84 | |
| p44 | DE + K360D_S364V | 0.69 | 1.02 | 73.50 | 24.79 | |
| p45 | DE + K360D_K409E | | | 90.43 | 9.57 | |
| p46 | DE + S364L_K409L | 0.29 | 0.71 | 93.71 | 5.29 | |
| p47 | DE + S364T_K409E | | 1.22 | 80.58 | 18.20 | |
| p48 | DE only | 0.3 | 1.47 | 93.46 | 4.76 | |

TABLE 7
Summary of HP-SEC results after the samples were heated
to 50° C. for 2 days

| | 50° C. | | | | |
|---|---|---|---|---|---|
| | | | | | Late |
| | Early Early (%) | Early (%) | Main | Late (%) | Late (%) |
| 1:1 | Aggregates | Aggregates | (%) IgG | Halfbody | Fragments |
| p33 | 1.01-7.32 − 4.51 | | 66.07 | 20.49 | 0.60 |
| p34 | 0.28 | 7.83 | 81.13 | 10.76 | |
| p35 | 0.37-5.76 − 4.18 | | 72.75 | 16.39 | 0.54 |
| p36 | 0.61 | 12.63 | 71.35 | 15.41 | |
| p37 | 0.31-5.09 − 3.56 | | 71.08 | 19.53 | 0.44 |
| p38 | 0.37 | 5.25 | 88.88 | 5.50 | |
| p39 | | 14 | 76.90 | 9.10 | |
| p40 | 7.20 | 22.33 | 64.57 | 5.07 | 0.82 |
| p47 | 9.18 | 6.72 | 66.44 | 16.76 | 0.91 |
| p42 | 0.48 | 7.48 | 72.40 | 18.84 | 0.80 |
| p43 | 0.72 | 7.54 | 74.55 | 16.52 | 0.67 |
| p44 | | 7.86 | 68.75 | 22.44 | 0.94 |
| p45 | | 9.81 | 81.37 | 7.48 | 1.34 |
| p46 | | 4.84 | 90.43 | 4.16 | 0.57 |
| p47 | 0.52 | 11.71 | 70.80 | 16.02 | 0.95 |
| p48 | 0.94 | 3.72 | 90.12 | 4.33 | 0.88 |

TABLE 8
Summary of HP-SEC results after the samples
were 5× freeze-thawed

| | Freeze/Thawed (5 times) | | | | |
|---|---|---|---|---|---|
| | Early Early | | | | Late |
| | (%) | Early (%) | Main (%) | Late (%) | Late (%) |
| 1:1 | Aggregates | Aggregates | IgG | Halfbody | Fragments |
| p33 | 0.60 | 1.05 | 73.71 | 24.64 | |
| p34 | | 0.32 | 87.59 | 12.08 | |
| p35 | 0.57-0.70 − 0.33 | | 78.25 | 20.15 | |
| p36 | 0.95 | 1.70 | 79.88 | 17.47 | |
| p37 | 0.68 | 1.45 | 76.54 | 21.33 | |
| p38 | | 1.24 | 93.24 | 5.52 | |
| p39 | | 1.34 | 85.48 | 11.58 | 1.60 |
| p40 | | 1.59 | 91.46 | 6.95 | |
| p47 | 0.26 | 0.56 | 78.60 | 20.58 | |
| p42 | | 1.86 | 75.89 | 22.25 | |
| p43 | 0.56 | 1.40 | 80.45 | 17.59 | |
| p44 | | 0.44 | 74.64 | 24.92 | |
| p45 | | 1.05 | 89.82 | 9.13 | |
| p46 | | 1.03 | 93.71 | 5.26 | |
| p47 | 0.73 | 0.92 | 79.73 | 18.61 | |
| p48 | 0.36 | 1.97 | 93.66 | 4.01 | |

CIEX-HPLC

The generated IgGs were analyzed on CIEX-HPLC. Agilent 1260 series HPLC system, Tosoh TSKgel SP-STAT column cat #21964, buffers used: A) 25 mM NaPO₄, pH 6.0 and B) 25 mM NaPO₄, 1M NaCl, pH 6.0, analysis with linear gradient of 0-30% buffer B (increase of 2% B per mL) at 0.5 mL/min, 10 μg of sample loaded. The generated profiles were analyzed for peak shape, retention times, bispecific/contaminants ratio and separation between bispecific and contaminants (most notably in 3:1 transfected samples). The separation between wt PG1122 and wt PG6744 is ˜3.7 min., which is increased by the KK/DE and other variants. WT DEKK ctrl. showed a peak at 10.25 (biclonic) and 7.8 minutes (DEDE homodimer), as expected. Ratios of either 1:1 or 1:3 productions have nearly identical retention time (RT) values.

Whilst some samples showed early-eluting species consisting of DEDE homodimers in one or both productions, other samples did not have any detectable early-eluting species.

Most notably, large amounts of early-eluting species were observed in the S354Q variant (both productions), S364T variant and WT (3:1 production) but all other samples had <10%.

All samples showed a pre-peak. Larger pre-peaks were observed in 1:3 productions, which was approximately 3 times larger than peaks observed in 1:1 production, compared to the height of the main peak. Without wishing to be bound to the theory, the inventors believe that the DE* halfbody species did not bind to the column and elutes in the “pre-peak” (the dead volume of the column), due to the additional negative charges that are present in some variants.

Many variants are very successful in preventing homodimer formation and as evident by the lack of early eluting peaks.

In summary, for 15 out of the 18 IgGs produced, only very small or non-existent amount of DEDE homodimers formed according to CIEX-HPLC, even in a 3:1 over-expression of the (modified) DE-arm. This indicates that most of the the additional variants are successful in preventing or mitigating homodimer formation (Table 9) as compared to the DEKK control.

TABLE 9
Summary of CIEX-HPLC results; RT = retention time, charge difference = additional (negative)
charge compared to original DE construct, empty field: no peak detected (1% threshold).

| | | | 1:1% | 3:1% | 1:1% | 3:1% | Charge | RT biclonic |
|---|---|---|---|---|---|---|---|---|
| Prod. 1:1 | Prod. 3:1 | Variant | DEDE | DEDE | DEKK | DEKK | difference | 1:1 (min.) |
| PB31852p33 | PB31852p49 | DE + S364V | 2.16 | 18.98 | 96.35 | 79.3 | 0 | 10.56 |
| PB31852p34 | PB31852p50 | DE + S364L | 0 | 0 | 97.86 | 96.91 | 0 | 10.73 |
| PB31852p35 | PB31852p51 | DE + S364I | 0 | 2.7 | 97.77 | 94.8 | 0 | 10.73 |
| PB31852p36 | PB31852p52 | DE + S364T | 11.45 | 49.57 | 86.57 | 48.87 | 0 | 10.28 |
| PB31852p37 | PB31852p53 | DE + K409I | 2.59 | 22.31 | 95.34 | 76.39 | −1 | 9.88 |
| PB31852p38 | PB31852p54 | DE + K409L | 1.64 | 9.76 | 97.43 | 89.66 | −1 | 9.84 |
| PB31852p39 | PB31852p55 | DE + K409E | 0 | 0 | 99.34 | 97.81 | −2 | 9.77 |
| PB31852p40 | PB31852p56 | DE + S354Q | 26.55 | 62.84 | 71.78 | 36.21 | 0 | 10.36 |
| PB31852p47 | PB31852p63 | DE + S364T_K409E | 0 | 0 | 98.68 | 98.76 | −2 | 9.8 |
| PB31852p42 | PB31852p58 | DE + K360D_S364I | 0 | 2.46 | 99.05 | 97.08 | −2 | 9.46 |
| PB31852p43 | PB31852p59 | DE + K360D_S364T | 2.38 | 9.26 | 96.78 | 60.3 | −2 | 9.51 |
| PB31852p44 | PB31852p60 | DE + K360D_S364V | 0 | 4.02 | 98.40 | 70.34 | −2 | 9.51 |
| PB31852p45 | PB31852p61 | DE + K360D_K409E | 0 | 0 | 96.7 | 97.35 | −4 | 9.25 |
| PB31852p46 | PB31852p62 | DE + S364L_K409L | 0 | 0 | 98.64 | 98.96 | −1 | 10.18 |
| PB31852p47 | PB31852p63 | DE + S364T_K409E | 0 | 0 | 97.2 | 97.43 | −2 | 9.79 |
| PB31852p48 | PB31852p64 | DE only | 2.27 | 62.89 | 95.76 | 34.66 | 0 | 10.25 |
| | | | | | | | Ratio | |
| | | | | Early | Early | | pre/ | |
| | | | RT biclonic | RT 1:1 | RT 3:1 | Ratio pre/ | highest | Ratio of |
| Prod. 1:1 | Prod. 3:1 | Variant | 3:1 (min.) | (min.) | (min.) | highest 1:1 | 1:3 | ratios |
| PB31852p33 | PB31852p49 | DE + S364V | 10.54 | 7.28 | 7.24 | 1.41 | 3.52 | 2.5 |
| PB31852p34 | PB31852p50 | DE + S364L | 10.72 | | | 0.33 | 0.85 | 2.6 |
| PB31852p35 | PB31852p51 | DE + S364I | 10.71 | | 7.38 | 0.87 | 2.91 | 3.3 |
| PB31852p36 | PB31852p52 | DE + S364T | 10.3 | 7.38 | 7.38 | 1.11 | 4.29 | 3.9 |
| PB31852p37 | PB31852p53 | DE + K409I | 9.85 | 6.63 | 6.62 | 1.82 | 6.85 | 3.8 |
| PB31852p38 | PB31852p54 | DE + K409L | 9.82 | 7.05 | 7.05 | 0.21 | 0.59 | 2.8 |
| PB31852p39 | PB31852p55 | DE + K409E | 9.75 | | | 0.45 | 0.86 | 1.9 |
| PB31852p40 | PB31852p56 | DE + S354Q | 10.32 | 7.14 | 7.13 | 0.12 | 0.18 | 1.5 |
| PB31852p47 | PB31852p63 | DE + S364T_K409E | 9.8 | | | 1.29 | 4.11 | 3.2 |
| PB31852p42 | PB31852p58 | DE + K360D_S364I | 9.46 | | 7.04 | 1.79 | 6.26 | 3.5 |
| PB31852p43 | PB31852p59 | DE + K360D_S364T | 9.48 | 5.9 | 5.85 | 1.52 | 5.67 | 3.7 |
| PB31852p44 | PB31852p60 | DE + K360D_S364V | 9.51 | | 5.66 | 2.18 | 7.19 | 3.3 |
| PB31852p45 | PB31852p61 | DE + K360D_K409E | 9.23 | | | 0.34 | 0.72 | 2.1 |
| PB31852p46 | PB31852p62 | DE + S364L_K409L | 10.21 | | | 0.16 | 0.52 | 3.2 |
| PB31852p47 | PB31852p63 | DE + S364T_K409E | 9.82 | | | 1.07 | 4.35 | 4.0 |
| PB31852p48 | PB31852p64 | DE only | 10.24 | 7.8 | 7.76 | 0.10 | 0.39 | 3.8 |

No DEDE homodimers detected
Small homodimer peak (0-5%)
Significant homodimer peak (5-25%)
Large homodimer peak (>25%)

LabCHIP

To determine the amount of IgG (DEKK+DEDE) and halfbody in the samples of Biclonic productions LabChip was used. Perkin Elmer LabCihp GXII Touch HT using Protein Clear HR Reagent kit (cat #CLS960014) and Protein Express Assay LabChip (cat #760499), analysis according to manufacturer's instructions, sample concentration 0.3 μg/μL. It was observed that DE halfbody formation differs significantly between variants and that the 3:1 production contains more DE halfbodies, as expected (Table 10).

TABLE 10
Summary of LabChip data

| | | | 1:1 | 1:1 | 3:1 | | |
|---|---|---|---|---|---|---|---|
| | | | IgG | halfbody | IgG | 3:1 | Ratio |
| 1:1 | 3:1 | Variant | (%) | (%) | (%) | halfbody | halfbody |
| p33 | p49 | DE + S364V | 77.1 | 22.9 | 44.1 | 38.28 | 1.67 |
| p34 | p50 | DE + S364L | 91.1 | 8.9 | 21.5 | 20.16 | 2.27 |
| p35 | p51 | DE + S364I | 83.4 | 16.6 | 41.2 | 33.3 | 2.01 |
| p36 | p52 | DE + S364T | 86.5 | 13.5 | 30 | 27.78 | 2.06 |
| p37 | p53 | DE + K409I | 82.6 | 17.4 | 48.9 | 42.78 | 2.46 |
| p38 | p54 | DE + K409L | 96.9 | 3.1 | 12.6 | 10.85 | 3.50 |
| p39 | p55 | DE + K409E | 91.7 | 8.3 | 14.4 | 14.31 | 1.72 |
| p40 | p56 | DE + S354Q | 95.8 | 4.2 | 5.7 | 6.36 | 1.51 |
| p47 | p63 | DE + S364T_K409E | 82.2 | 17.8 | 43.9 | 37.44 | 2.10 |
| p42 | p58 | DE + K360D_S364I | 82.2 | 17.8 | 47.3 | 35.3 | 1.98 |
| p43 | p59 | DE + K360D_S364T | 86.1 | 13.9 | 29.8 | 22.62 | 1.63 |
| p44 | p60 | DE + K360D_S364V | 78.7 | 21.3 | 45.2 | 41.84 | 1.96 |
| p45 | p61 | DE + K360D_K409E | 93.1 | 6.9 | 10.5 | 12.5 | 1.81 |
| p46 | p62 | DE + S364L_K409L | 97 | 3 | 8.1 | 20.87 | 6.96 |
| p47 | p63 | DE + S364T_K409E | 83.3 | 16.7 | 43.1 | 39.42 | 2.36 |
| p48 | p64 | DE only | 100 | 0 | 7.9 | 8.51 | N/A |

nMS

For all generated IgGs, 100 μg/sample was analyzed in nMS analysis (see methodology in Rosati et al, Anal Chem. 2012 Aug. 21; 84(16):7227-32). The relative ratios between the various species (bispecific, homodimers, half bodies) were studied.

All 16 samples were successfully analyzed, and IgG species could be identified by this technique. The control DEKK sample resulted in 95% bispecific in a 1:1 production. Percentages of DEKK variants heterodimer formation in 1:1 production varied from 74% to 100%. A total of 12 samples contained >90% DEKK heterodimer. Homodimer formation was significantly reduced in 14 samples as compared to the DEKK control. For 8 of the samples 2% DEDE homodimers was formed in the 3:1 production.

In summary, 14 of the variants significantly reduced homodimer formation in 3:1 production (as compared to a DEKK control) and for 8 variants almost no homodimer was formed (Table 11).

TABLE 11
Summary of nMS results

| | | | 1:1 DEKK | 3:1 DEKK | 1:1 DEDE | 3:1 DEDE | 1:1 DE | 3:1 DE |
|---|---|---|---|---|---|---|---|---|
| Prod. 1:1 | Prod. 3:1 | Variant | (% of total) | (% of total) | (% of total) | (% of total) | (% of total) | (% of total) |
| PB31852p33 | PB31852p49 | DE + S364V | 85 | 57 | 4 | 9 | 10 | 34 |
| PB31852p34 | PB31852p50 | DE + S364L | 98 | 93 | 2 | 2 | 0 | 5 |
| PB31852p35 | PB31852p51 | DE + S364I | 93 | 82 | 1 | 2 | 6 | 16 |
| PB31852p36 | PB31852p52 | DE + S364T | 85 | 51 | 7 | 31 | 8 | 18 |
| PB31852p37 | PB31852p53 | DE + K409I | 87 | 59 | 2 | 7 | 11 | 34 |
| PB31852p38 | PB31852p54 | DE + K409L | 93 | 93 | 7 | 2 | 0 | 5 |
| PB31852p39 | PB31852p55 | DE + K409E | 99 | 97 | 1 | 1 | 0 | 2 |
| PB31852p40 | PB31852p56 | DE + S354Q | 74 | 44 | 26 | 55 | 0 | 1 |
| PB31852p47 | PB31852p63 | DE + S364T_K409E | 90 | 78 | 2 | 1 | 8 | 22 |
| PB31852p42 | PB31852p58 | DE + K360D_S364I | 92 | 61 | 2 | 5 | 6 | 34 |
| PB31852p43 | PB31852p59 | DE + K360D_S364T | 83 | 46 | 8 | 20 | 9 | 34 |
| PB31852p44 | PB31852p60 | DE + K360D_S364V | 83 | 62 | 5 | 5 | 12 | 34 |
| PB31852p45 | PB31852p61 | DE + K360D_K409E | 99 | 94 | 1 | 1 | 0 | 5 |
| PB31852p46 | PB31852p62 | DE + S364L_K409L | 100 | 96 | 0 | 2 | 0 | 2 |
| PB31852p47 | PB31852p63 | DE + S364T_K409E | 95 | 79 | 0 | 2 | 5 | 20 |
| PB31852p48 | PB31852p64 | DE only | 95 | 42 | 5 | 56 | 0 | 2 |

% of DEDE: Non-/barely detectable peak (~<5%)
% of DEDE: Clearly detectable peak (~5-10%)
% of DEDE: Significant peak (10-25%)
% of DEDE: Significant/large peak (~>25%)

Summary of Results

Different methods were used to detect halfbodies: LabChip, HP-SEC and nMS. Whilst the values obtained differ in absolute terms and nMS detects less halfbodies than other methods, the ultimate conclusion concerning the utility of the variants at reducing DEDE homodimer formation was consistent (Table 12). Different techniques were used to characterize the samples for the presence of DEKK, IgG-derived contaminants and aggregates, and whilst the values differ in absolute terms, consistent conclusions could be drawn from the obtained data (Tables 13-15).

TABLE 12
Overview of halfbody formation determination
by different experimental methods

| | LabChip % | HP-SEC % | nMS ~% |
|---|---|---|---|
| Variant | halfbody 3:1 | halfbody 3:1 | halfbody 3:1 |
| DE + S364V | 38.28 | 41.63 | 27.7 |
| DE + S364L | 20.16 | 20.6 | 16.8 |
| DE + S364I | 33.3 | 38.87 | 25.0 |
| DE + S364T | 27.78 | 30.36 | 21.7 |
| DE + K409I | 42.78 | 44.85 | 30.0 |
| DE + K409L | 10.85 | 14.94 | 9.8 |
| DE + K409E | 14.31 | 16.58 | 12.5 |
| DE + S354Q | 6.36 | 7.66 | 6.0 |
| DE + S364T_K409E | 37.44 | 40.26 | 27.2 |
| DE + K360D_S364I | 35.3 | 44.37 | 26.1 |
| DE + K360D_S364T | 22.62 | 31.01 | 18.4 |
| DE + K360D_S364V | 41.84 | 44.11 | 29.5 |
| DE + K360D_K409E | 12.5 | 13.39 | 11.1 |
| DE + S364L_K409L | 20.87 | 9.96 | 17.3 |
| DE + S364T_K409E | 39.42 | 40.85 | 28.3 |
| DE only | 8.51 | 11.02 | 7.8 |

TABLE 13
Overview of homodimer formation as determined by CIEX and nMS

| | | | 1:1% | 1:1% | 3:1% | 3:1% |
|---|---|---|---|---|---|---|
| | | | DEKK | DEKK | DEKK | DEKK |
| 1:1 | 1:3 | Variant | (nMS) | (CI EX) | (nMS) | (CIEX) |
| p33 | p49 | DE + S364V | 85 | 96.35 | 57 | 79.3 |
| p34 | p50 | DE + S364L | 98 | 97.86 | 93 | 96.91 |
| p35 | p51 | DE + S364I | 93 | 97.77 | 82 | 94.8 |
| p36 | p52 | DE + S364T | 85 | 86.57 | 51 | 48.87 |
| p37 | p53 | DE + K409I | 87 | 95.34 | 59 | 76.39 |
| p38 | p54 | DE + K409L | 93 | 97.43 | 93 | 89.66 |
| p39 | p55 | DE + K409E | 99 | 99.34 | 97 | 97.81 |
| p40 | p56 | DE + S354Q | 74 | 71.78 | 44 | 36.21 |
| p47 | p63 | DE + S364T_K409E | 90 | 98.68 | 78 | 98.76 |
| p42 | p58 | DE + K360D_S364I | 92 | 99.05 | 61 | 97.08 |
| p43 | p59 | DE + K360D_S364T | 83 | 96.78 | 46 | 60.3 |
| p44 | p60 | DE + K360D_S364V | 83 | 98.40 | 62 | 70.34 |
| p45 | p61 | DE + K360D_K409E | 99 | 96.7 | 94 | 97.35 |
| p46 | p62 | DE + S364L_K409L | 100 | 98.64 | 96 | 98.96 |
| p47 | p63 | DE + S364T_K409E | 95 | 97.2 | 79 | 97.43 |
| p48 | p64 | DE only | 95 | 95.76 | 42 | 34.66 |

TABLE 14
Overview of homodimer formation as determined by CIEX and nMS

| | | | % DEDE in 1:1 | | % DEDE in 3:1 | % DEDE in |
|---|---|---|---|---|---|---|
| | | | prod. (CIEX- | % DEDE in 1:1 | prod. (CIEX- | 3:1 prod. |
| | 1:1 | 1:3 | HPLC) | prod. (nMS) | HPLC) | (nMS) |
| DE + S364V | p33 | p49 | 2.2 | 4 | 19.0 | 9 |
| DE + S364L | p34 | p50 | 0.0 | 2 | 0.0 | 2 |
| DE + S364I | p35 | p51 | 0.0 | 1 | 2.7 | 2 |
| DE + S364T | p36 | p52 | 11.5 | 7 | 49.6 | 31 |
| DE + K409I | p37 | p53 | 2.6 | 2 | 22.3 | 7 |
| DE + K409L | p38 | p54 | 1.6 | 7 | 9.8 | 2 |
| DE + K409E | p39 | p55 | 0.0 | 1 | 0.0 | 1 |
| DE + S354Q | p40 | p56 | 26.6 | 26 | 62.8 | 55 |
| DE + S364T_K409E | p47 | p63 | 0.0 | 2 | 0.0 | 1 |
| DE + K360D_S364I | p42 | p58 | 0.0 | 2 | 2.5 | 5 |
| DE + K360D_S364T | p43 | p59 | 2.4 | 8 | 9.3 | 20 |
| DE + K360D_S364V | p44 | p60 | 0.0 | 5 | 4.0 | 5 |
| DE + K360D_K409E | p45 | p61 | 0.0 | 1 | 0.0 | 1 |
| DE + S364L_K409L | p46 | p62 | 0.0 | 0 | 0.0 | 2 |
| DE + S364T_K409E | p47 | p63 | 0.0 | 0 | 0.0 | 2 |
| DE only | p48 | p64 | 2.3 | 5 | 62.9 | 56 |

| | | | Non/barely |
|---|---|---|---|
| No homodimer | Non/barely | No homodimer | detectable |
| peak | detectable peak | peak | peak |
| Homodimer | Detectable peak | Homodimer peak | Detectable |
| peak 0-5% | (~5-10%) | 0-5% | peak (~5-10%) |
| Homodimer | Significant peak | Homodimer peak | Significant |
| peak 5-25% | (~10-25%) | 5-25% | peak (-10-25%) |
| Homodimer | Large peak | Homodimer peak | Large peak |
| peak > 25% | (>25%) | > 25% | (>25%) |

TABLE 15
Overview 0 stability analysis as determined by DLS and HP-SEC

| | | | TM (Uncle, | DLS (Uncle, | HP-SEC | HP-SEC 2d | HP-SEC, 5× |
|---|---|---|---|---|---|---|---|
| | | | 1:1 | stressed | (1:1, 1xFT | @50 C. (% | FT (% |
| Variant | 1:1 | 1:3 | productions) | samples) | aggregates) | aggregates) | aggregates) |
| DE + S364V | p33 | p49 | 64.0 | Agg./fragments | 0 | 12.8 | 1.7 |
| | | | | detected (intensity | | | |
| | | | | only) | | | |
| DE + S364L | p34 | p50 | 63.0 | No aggr. Detected | 0 | 8.1 | 0.3 |
| DE + S364I | p35 | p51 | 64.1 | No aggr. Detected | 0 | 10.3 | 1.6 |
| DE + S364T | p36 | p52 | 65.7 | Agg./fragments | 0 | 13.2 | 2.7 |
| | | | | detected (intensity | | | |
| | | | | only) | | | |
| DE + K409I | p37 | p53 | 65.0 | No aggr. Detected | 2.3 | 9.0 | 2.1 |
| DE + K409L | p38 | p54 | 66.0 | No aggr. Detected | 0.5 | 5.6 | 1.2 |
| DE + K409E | p39 | p55 | 62.5 | No aggr. Detected | 0.5 | 14.0 | 1.3 |
| DE + S354Q | p40 | p56 | 64.5 | No aggr. Detected | 1.2 | 29.5 | 1.6 |
| DE + S364T_K409E | p47 | p63 | 62.5 | No aggr. Detected | 0.5 | 15.9 | 0.8 |
| DE + K360D_S364I | p42 | p58 | 64.7 | No aggr. Detected | 0.8 | 8.0 | 1.9 |
| DE + K360D_S364T | p43 | p59 | 65.5 | Agg./fragments | 2.2 | 8.3 | 2.0 |
| | | | | detected (intensity | | | |
| | | | | only) | | | |
| DE + K360D_S364V | p44 | p60 | 64.2 | No aggr. Detected | 1.6 | 7.9 | 0.4 |
| DE + K360D_K409E | p45 | p61 | 62.5 | No aggr. Detected | 2.0 | 9.8 | 1.1 |
| DE + S364L_K409L | p46 | p62 | 65.1 | No aggr. Detected | 0.7 | 4.8 | 1.0 |
| DE + S364T_K409E | p47 | p63 | 64.0 | No aggr. Detected | 0.4 | 12.2 | 1.7 |
| DE only | p48 | p64 | 67.7 | No aggr. Detected | 1.5 | 4.7 | 2.3 |

In summary, the variants provided herein increase heterodimer stability and reduce homodimer formation based on variations at the CH3 interface of the heterodimeric polypeptide.

### Example 4

The influence of variants listed in Table 16 on DEKK heterodimer formation in terms of yield, purity and formation of DE contaminants, if any, was further investigated. In this specific example, bispecific proteins carrying variants in the DE heavy chain were produced together with the KK-comprising heavy chain in varying DE:KK ratios as mentioned, purified using Protein-A and analyzed by CIEX and analyzed by LabChip/SDS-PAGE.

Cloning:

VH sequences for anti-TT on the DE side and anti-CD3 on the KK side, were cloned using SfiI and XhoI into vectors containing the following variants: K360D_K409E (SEQ ID NO: 12), S364L_K409L (SEQ ID NO: 14), S364L (SEQ ID NO: 2) and S364T_K409E (SEQ ID NO: 13)). Colony/sequence PCR was performed on the ligation products in order to retrieve all variants. All confirmed variants were miniprepped, followed by sequencing. IgGs were produced by transfections of single DE* constructs (labelled PGxxxx IgG) and DE* constructs combined with KK constructs (labelled PBxxxxx bispecific IgG) at varying DE:KK ratios (Table 16) into 293FF cells. After 7 days of incubation (37° C., 8% CO2, 155 rpm) cell supernatant was harvested, followed by Protein A purification (acidic elution) and buffer exchange to PBS pH7.4. The IgG concentration of the samples was measured. The proteins were subsequently characterized by HP-SEC, CIEX and Lab-Chip/SDS-PAGE.

TABLE 16
Production of bispecifics and
single arms on 24-well format.

| # | variant | ratio DE:KK |
|---|---|---|
| 1 | DEKK | |
| 2 | DEKK | 2:1 |
| 3 | DEKK | 1:1 |
| 4 | DEKK | 1:2 |
| 5 | DEKK | |
| 6 | K360D K409E | |
| 7 | K360D K409E | 2:1 |
| 8 | K360D K409E | 1:1 |
| 9 | K360D K409E | 1:2 |
| 10 | S364L K409L | |
| 11 | S364L K409L | 2:1 |
| 12 | S364L K409L | 1:1 |
| 13 | S364L K409L | 1:2 |
| 14 | S364L | |
| 15 | S364L | 2:1 |
| 16 | S364L | 1:1 |
| 17 | S364L | 1:2 |
| 18 | S364T K409E | |
| 19 | S364T K409E | 2:1 |
| 20 | S364T K409E | 1:1 |
| 21 | S364T K409E | 1:2 |

HP-SEC:

Single arm productions were analyzed by HP-SEC, according to protocol described in Example 3. Similar amount of each IgG, but not less than 10 ug, were used. For all variants the DE arm runs as >75% half-body, ˜10-20% homodimer and some aggregates, under native conditions (Table 17).

TABLE 17
HP-SEC analysis of single arm productions

| | HP-SEC % | | |
|---|---|---|---|
| Variant | Halfbody | Homodimer | Aggregate |
| WT-DE | 8.8 | 88.9 | 2.2 |
| S364T K409E | 75.6 | 23.5 | 0.9 |
| K360D K409E | 76.0 | 23.3 | 0.6 |
| S364L | 81.4 | 16.6 | 2.0 |
| S364L K409L | 85.9 | 12.5 | 1.6 |

CIEX Analysis:

IgG proteins were analyzed by CIEX (protocol described in Example 3). As control, single arm productions were analyzed to be able to assign the detected peaks in bispecific samples. Protein amount injected was adjusted for all samples. The protein amount of samples containing a single arm was adjusted to 5 ug for each sample. The maximum possible volume (100 uL) was injected in case concentrations were too low. The expected retention time (RT) of WT-DE homodimer with an anti-TT heavy chain (VH; SEQ ID NO: 16) is 13 min; the RT of KK halfbody with anti-CD3 heavy chain (VH; SEQ ID NO: 17) is 26 min. The retention time of the KK-KK dimer is such that it elutes too late for it to be detected. The pre-peak was not taken into account for quantification of the components in the samples. Peaks that belong to the same species (PB, DE or KK) were summed up and added as one value. The composition of sample eluting from the CIEX column is based on the percentage of area of each peak.

The purity of the heterodimers improved for all investigated variants under tested conditions when comparing the same ratios (Table 18). The effect of changing the DE:KK ratio was such that an increase in KK DNA, and thereby increased translation of KK polypeptide, resulted in decrease of the DE contaminant.

TABLE 18
CIEX analysis of single arm productions

| Variant | Ratio DE:KK | % PB | % DE | % KK |
|---|---|---|---|---|
| DEKK | 2:1 | 64 | 36 | 0 |
| | 1:1 | 76 | 17 | 7 |
| | 1:2 | 73 | 7 | 20 |
| K360D K409E | 2:1 | 92 | 0 | 8 |
| | 1:1 | 75 | 0 | 24 |
| | 1:2 | 59 | 0 | 40 |
| S364L K409L | 2:1 | 95 | 0 | 5 |
| | 1:1 | 86 | 0 | 14 |
| | 1:2 | 58 | 0 | 40 |
| S364L | 2:1 | 88 | 0 | 12 |
| | 1:1 | 73 | 0 | 27 |
| | 1:2 | 53 | 0 | 46 |
| S364T K409E | 2:1 | 92 | 0 | 8 |
| | 1:1 | 77 | 0 | 22 |
| | 1:2 | 54 | 0 | 46 |

In single arm productions, DEDE homodimer of variant decreased and DE halfbody increased (Table 19). The results are consistent with data generated using HP-SEC.

TABLE 19
LabChip/SDS-PAGE for Single arm productions

| CH3 | Sample | variant | Halfbody | Homodimer |
|---|---|---|---|---|
| DE | PG1516p47 | WT | 11.7 | 88.3 |
| DE | PG1516p46 | S364T | 82.2 | 17.8 |
| | | K409E | | |
| DE | PG1516p43 | K360D | 83.9 | 16.1 |
| | | K409E | | |
| DE | PG1516p45 | S364L | 89.2 | 10.8 |
| DE | PG1516p44 | S364L | 91.8 | 8.2 |
| | | K409L | | |
| KK | PG3896p12 | WT | 100 | 0 |

Influence of Variants on Purity

The amount of bispecific protein present in each sample was estimated using CIEX results to obtain insight in the purity of the bispecific proteins. The purity of heterodimer PB protein is improved in this example which includes Protein A purification. Purity is improved by increasing the DE:KK ratio (Table 20)

TABLE 20
Influence of tested variants on purity

| Variant | Ratio DE:KK | % PB | % DE | % KK |
|---|---|---|---|---|
| DEKK | 2:1 | 64 | 36 | 0 |
| | 1:1 | 76 | 17 | 7 |
| | 1:2 | 73 | 7 | 20 |
| K360D K409E | 2:1 | 92 | 0 | 8 |
| | 1:1 | 75 | 0 | 24 |
| | 1:2 | 59 | 0 | 40 |
| S364L K409L | 2:1 | 95 | 0 | 5 |
| | 1:1 | 86 | 0 | 14 |
| | 1:2 | 58 | 0 | 40 |
| S364L | 2:1 | 88 | 0 | 12 |
| | 1:1 | 73 | 0 | 27 |
| | 1:2 | 53 | 0 | 46 |
| S364T K409E | 2:1 | 92 | 0 | 8 |
| | 1:1 | 77 | 0 | 22 |
| | 1:2 | 54 | 0 | 46 |

Sequences used:

>MV1708_DE_S364V
[SEQ ID NO: 1]
TCCGGAACTCCTGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCA
AGGTTTCGAACAAAGCCCTCCCAGCCCCCATCGAGAAAACCATCTCCAAAGCCAAAGGGCAGCCCCGA
GAACCACAGGTGTACACCGACCCCCCATCCCGGGAGGAGATGACCAAGAACCAGGTCGTCCTGACCTG
CGAGGTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCAAGCTCACCGTG
GACAAGAGCAGGTGGCAGCAGGGGAACGTCTTCTCATGCTCCGTGATGCATGAGGCTCTGCACAACCA
CTACACGCAGAAGAGCCTCTCCCTGTCTCCGGGTAAATGAGTTTAACGGATCTTAATTAATCCGAGCT
CGGTACCAAGCTTAAG
>MV1708_DE_S364L
[SEQ ID NO: 2]
TCCGGAACTCCTGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCA
AGGTTTCGAACAAAGCCCTCCCAGCCCCCATCGAGAAAACCATCTCCAAAGCCAAAGGGCAGCCCCGA
GAACCACAGGTGTACACCGACCCCCCATCCCGGGAGGAGATGACCAAGAACCAGGTCCTCCTGACCTG
CGAGGTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCAAGCTCACCGTG
GACAAGAGCAGGTGGCAGCAGGGGAACGTCTTCTCATGCTCCGTGATGCATGAGGCTCTGCACAACCA
CTACACGCAGAAGAGCCTCTCCCTGTCTCCGGGTAAATGAGTTTAACGGATCTTAATTAATCCGAGCT
CGGTACCAAGCTTAAG
>MV1708_DE_S364I
[SEQ ID NO: 3]
TCCGGAACTCCTGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCA
AGGTTTCGAACAAAGCCCTCCCAGCCCCCATCGAGAAAACCATCTCCAAAGCCAAAGGGCAGCCCCGA
GAACCACAGGTGTACACCGACCCCCCATCCCGGGAGGAGATGACCAAGAACCAGGTCATCCTGACCTG
CGAGGTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCAAGCTCACCGTG
GACAAGAGCAGGTGGCAGCAGGGGAACGTCTTCTCATGCTCCGTGATGCATGAGGCTCTGCACAACCA
CTACACGCAGAAGAGCCTCTCCCTGTCTCCGGGTAAATGAGTTTAACGGATCTTAATTAATCCGAGCT
CGGTACCAAGCTTAAG
>MV1708_DE_S364T
[SEQ ID NO: 4]
TCCGGAACTCCTGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCA
AGGTTTCGAACAAAGCCCTCCCAGCCCCCATCGAGAAAACCATCTCCAAAGCCAAAGGGCAGCCCCGA
GAACCACAGGTGTACACCGACCCCCCATCCCGGGAGGAGATGACCAAGAACCAGGTCACACTGACCTG
CGAGGTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCAAGCTCACCGTG
GACAAGAGCAGGTGGCAGCAGGGGAACGTCTTCTCATGCTCCGTGATGCATGAGGCTCTGCACAACCA
CTACACGCAGAAGAGCCTCTCCCTGTCTCCGGGTAAATGAGTTTAACGGATCTTAATTAATCCGAGCT
CGGTACCAAGCTTAAG
>MV1708_DE_S364Q
[SEQ ID NO: 5]
TCCGGAACTCCTGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCA
AGGTTTCGAACAAAGCCCTCCCAGCCCCCATCGAGAAAACCATCTCCAAAGCCAAAGGGCAGCCCCGA
GAACCACAGGTGTACACCGACCCCCCATCCCGGGAGGAGATGACCAAGAACCAGGTCCAGCTGACCTG
CGAGGTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCAAGCTCACCGTG
GACAAGAGCAGGTGGCAGCAGGGGAACGTCTTCTCATGCTCCGTGATGCATGAGGCTCTGCACAACCA
CTACACGCAGAAGAGCCTCTCCCTGTCTCCGGGTAAATGAGTTTAACGGATCTTAATTAATCCGAGCT
CGGTACCAAGCTTAAG
>MV1708_DE_K409I
[SEQ ID NO: 6]
TCCGGAACTCCTGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCA
AGGTTTCGAACAAAGCCCTCCCAGCCCCCATCGAGAAAACCATCTCCAAAGCCAAAGGGCAGCCCCGA
GAACCACAGGTGTACACCGACCCCCCATCCCGGGAGGAGATGACCAAGAACCAGGTCAGCCTGACCTG
CGAGGTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCATCCTCACCGTG
GACAAGAGCAGGTGGCAGCAGGGGAACGTCTTCTCATGCTCCGTGATGCATGAGGCTCTGCACAACCA
CTACACGCAGAAGAGCCTCTCCCTGTCTCCGGGTAAATGAGTTTAACGGATCTTAATTAATCCGAGCT
CGGTACCAAGCTTAAG
>MV1708_DE_K409L
[SEQ ID NO: 7]
TCCGGAACTCCTGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCA
AGGTTTCGAACAAAGCCCTCCCAGCCCCCATCGAGAAAACCATCTCCAAAGCCAAAGGGCAGCCCCGA
GAACCACAGGTGTACACCGACCCCCCATCCCGGGAGGAGATGACCAAGAACCAGGTCAGCCTGACCTG
CGAGGTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCCTGCTCACCGTG
GACAAGAGCAGGTGGCAGCAGGGGAACGTCTTCTCATGCTCCGTGATGCATGAGGCTCTGCACAACCA
CTACACGCAGAAGAGCCTCTCCCTGTCTCCGGGTAAATGAGTTTAACGGATCTTAATTAATCCGAGCT
CGGTACCAAGCTTAAG
>MV1708_DE_K409E
[SEQ ID NO: 8]
TCCGGAACTCCTGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCA
AGGTTTCGAACAAAGCCCTCCCAGCCCCCATCGAGAAAACCATCTCCAAAGCCAAAGGGCAGCCCCGA
GAACCACAGGTGTACACCGACCCCCCATCCCGGGAGGAGATGACCAAGAACCAGGTCAGCCTGACCTG
CGAGGTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCGAACTCACCGTG
GACAAGAGCAGGTGGCAGCAGGGGAACGTCTTCTCATGCTCCGTGATGCATGAGGCTCTGCACAACCA
CTACACGCAGAAGAGCCTCTCCCTGTCTCCGGGTAAATGAGTTTAACGGATCTTAATTAATCCGAGCT
CGGTACCAAGCTTAAG
>CH2-3_DE_K360D-S364I
[SEQ ID NO: 9]
Tccggaactcctggggggaccgtcagtcttcctcttccccccaaaacccaaggacaccctcatgatct
cccggacccctgaggtcacatgcgtggtggtggacgtgagccacgaagaccctgaggtcaagttcaac
tggtacgtggacggcgtggaggtgcataatgccaagacaaagccgcgggaggagcagtacaacagcac
gtaccgtgtggtcagcgtcctcaccgtcctgcaccaggactggctgaatggcaaggagtacaagtgca
aggtttcgaacaaagccctcccagcccccatcgagaaaaccatctccaaagccaaagggcagccccga
gaaccacaggtgtacaccgaccccccatcccgggaggagatgaccgacaaccaggtcatcctgacctg
cgaggtcaaaggcttctatcccagcgacatcgccgtggagtgggagagcaatgggcagccggagaaca
actacaagaccacgcctcccgtgctggactccgacggctccttcttcctctatagcaagctcaccgtg
gacaagagcaggtggcagcaggggaacgtcttctcatgctccgtgatgcatgaggctctgcacaacca
ctacacgcagaagagcctctccctgtctccgggtaaatgagtttaacggatcttaattaatccgagct
cggtaccaagcttaag
>CH2-3_DE_K360D-S364T
[SEQ ID NO: 10]
Tccggaactcctggggggaccgtcagtcttcctcttccccccaaaacccaaggacaccctcatgatct
cccggacccctgaggtcacatgcgtggtggtggacgtgagccacgaagaccctgaggtcaagttcaac
tggtacgtggacggcgtggaggtgcataatgccaagacaaagccgcgggaggagcagtacaacagcac
gtaccgtgtggtcagcgtcctcaccgtcctgcaccaggactggctgaatggcaaggagtacaagtgca
aggtttcgaacaaagccctcccagcccccatcgagaaaaccatctccaaagccaaagggcagccccga
gaaccacaggtgtacaccgaccccccatcccgggaggagatgaccaagaaccaggtcctcctgacctg
cgaggtcaaaggcttctatcccagcgacatcgccgtggagtgggagagcaatgggcagccggagaaca
actacaagaccacgcctcccgtgctggactccgacggctccttcttcctctatagcctgctcaccgtg
gacaagagcaggtggcagcaggggaacgtcttctcatgctccgtgatgcatgaggctctgcacaacca
ctacacgcagaagagcctctccctgtctccgggtaaatgagtttaacggatcttaattaatccgagct
cggtaccaagcttaag
>CH2-3_DE_K360D-S364V
[SEQ ID NO: 11]
Tccggaactcctggggggaccgtcagtcttcctcttccccccaaaacccaaggacaccctcatgatct
cccggacccctgaggtcacatgcgtggtggtggacgtgagccacgaagaccctgaggtcaagttcaac
tggtacgtggacggcgtggaggtgcataatgccaagacaaagccgcgggaggagcagtacaacagcac
gtaccgtgtggtcagcgtcctcaccgtcctgcaccaggactggctgaatggcaaggagtacaagtgca
aggtttcgaacaaagccctcccagcccccatcgagaaaaccatctccaaagccaaagggcagccccga
gaaccacaggtgtacaccgaccccccatcccgggaggagatgaccaagaaccaggtcctcctgacctg
cgaggtcaaaggcttctatcccagcgacatcgccgtggagtgggagagcaatgggcagccggagaaca
actacaagaccacgcctcccgtgctggactccgacggctccttcttcctctatagcctgctcaccgtg
gacaagagcaggtggcagcaggggaacgtcttctcatgctccgtgatgcatgaggctctgcacaacca
ctacacgcagaagagcctctccctgtctccgggtaaatgagtttaacggatcttaattaatccgagct
cggtaccaagcttaag
>CH2-3_DE_K360D-K409E
[SEQ ID NO: 12]
Ccggaactcctggggggaccgtcagtcttcctcttccccccaaaacccaaggacaccctcatgatctc
ccggacccctgaggtcacatgcgtggtggtggacgtgagccacgaagaccctgaggtcaagttcaact
ggtacgtggacggcgtggaggtgcataatgccaagacaaagccgcgggaggagcagtacaacagcacg
taccgtgtggtcagcgtcctcaccgtcctgcaccaggactggctgaatggcaaggagtacaagtgcaa
ggtttcgaacaaagccctcccagcccccatcgagaaaaccatctccaaagccaaagggcagccccgag
aaccacaggtgtacaccgaccccccatcccgggaggagatgaccgacaaccaggtcagcctgacctgc
gaggtcaaaggcttctatcccagcgacatcgccgtggagtgggagagcaatgggcagccggagaacaa
ctacaagaccacgcctcccgtgctggactccgacggctccttcttcctctatagcgagctcaccgtgg
acaagagcaggtggcagcaggggaacgtcttctcatgctccgtgatgcatgaggctctgcacaaccac
tacacgcagaagagcctctccctgtctccgggtaaatgagtttaacggatcttaattaatccgagctc
ggtaccaagcttaag
>CH2-3_DE_S364T-K409E
[SEQ ID NO: 13]
tccggaactcctggggggaccgtcagtcttcctcttccccccaaaacccaaggacaccctcatgatct
cccggacccctgaggtcacatgcgtggtggtggacgtgagccacgaagaccctgaggtcaagttcaac
tggtacgtggacggcgtggaggtgcataatgccaagacaaagccgcgggaggagcagtacaacagcac
gtaccgtgtggtcagcgtcctcaccgtcctgcaccaggactggctgaatggcaaggagtacaagtgca
aggtttcgaacaaagccctcccagcccccatcgagaaaaccatctccaaagccaaagggcagccccga
gaaccacaggtgtacaccgaccccccatcccgggaggagatgaccaagaaccaggtcacactgacctg
cgaggtcaaaggcttctatcccagcgacatcgccgtggagtgggagagcaatgggcagccggagaaca
actacaagaccacgcctcccgtgctggactccgacggctccttcttcctctatagcgagctcaccgtg
gacaagagcaggtggcagcaggggaacgtcttctcatgctccgtgatgcatgaggctctgcacaacca
ctacacgcagaagagcctctccctgtctccgggtaaatgagtttaacggatcttaattaatccgagct
cggtaccaagcttaag
>CH2-3_DE_S364L-K409L
[SEQ ID NO: 14]
tccggaactcctggggggaccgtcagtcttcctcttccccccaaaacccaaggacaccctcatgatct
cccggacccctgaggtcacatgcgtggtggtggacgtgagccacgaagaccctgaggtcaagttcaac
tggtacgtggacggcgtggaggtgcataatgccaagacaaagccgcgggaggagcagtacaacagcac
gtaccgtgtggtcagcgtcctcaccgtcctgcaccaggactggctgaatggcaaggagtacaagtgca
aggtttcgaacaaagccctcccagcccccatcgagaaaaccatctccaaagccaaagggcagccccga
gaaccacaggtgtacaccgaccccccatcccgggaggagatgaccaagaaccaggtcctcctgacctg
cgaggtcaaaggcttctatcccagcgacatcgccgtggagtgggagagcaatgggcagccggagaaca
actacaagaccacgcctcccgtgctggactccgacggctccttcttcctctatagcctgctcaccgtg
gacaagagcaggtggcagcaggggaacgtcttctcatgctccgtgatgcatgaggctctgcacaacca
ctacacgcagaagagcctctccctgtctccgggtaaatgagtttaacggatcttaattaatccgagct
cggtaccaagcttaag
>MV1708_DE_K360D
[SEQ ID NO: 15]
TCCGGAACTCCTGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCA
AGGTTTCGAACAAAGCCCTCCCAGCCCCCATCGAGAAAACCATCTCCAAAGCCAAAGGGCAGCCCCGA
GAACCACAGGTGTACACCGACCCCCCATCCCGGGAGGAGATGACCGACAACCAGGTCAGCCTGACCTG
CGAGGTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCAAGCTCACCGTG
GACAAGAGCAGGTGGCAGCAGGGGAACGTCTTCTCATGCTCCGTGATGCATGAGGCTCTGCACAACCA
CTACACGCAGAAGAGCCTCTCCCTGTCTCCGGGTAAATGAGTTTAACGGATCTTAATTAATCCGAGCT
CGGTACCAAGCTTAAG
>VH sequence of anti-TT Fab
[SEQ ID NO: 16]
EVQLVETGGGVVQPGRSLRLSCAASGFTFSQYAMHWVRQAPGKGLEWVAIISHDERNKYYVDSGMGRF
TISRDNSKNTLFLQMNSLRSEDTAVYYCARDMRKGGYYYGFDVWGQGTTVTVSS
>VH sequence of anti-CD3 Fab
[SEQ ID NO: 17]
QVQLVESGGGVVQPGRSLRLSCAASGFTFRSYGMHWVRQAPGKGLEWVAIIWYSGSKKNYADSVKGRF
TISRDNSKNTLYLQMNSLRAEDTAVYYCARGTGYNWFDPWGQGTLVTVSS

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. An isolated heterodimeric protein comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, and wherein the first human IgG CH3-containing polypeptide comprises a further amino acid variant at position S364, K409 and/or K360.

2. The isolated heterodimeric protein of claim 1, wherein:
a) the 364 amino acid is valine, isoleucine, threonine, glutamine or leucine; and/or
b) the 409 amino acid is isoleucine, leucine or glutamate; and/or
c) the 360 amino acid is aspartate.

3. The isolated heterodimeric protein of claim 1 or 2, wherein the heterodimeric protein comprises a human IgG1, an IgG2, an IgG3 or an IgG4 CH3-region.

4. The isolated heterodimeric protein of any one of claims 1 to 3, wherein the heterodimeric protein comprises a human immunoglobulin Fc region, optionally wherein the human immunoglobulin Fc region comprises an IgG1, an IgG2, an IgG3, or an IgG4 Fc region.

5. The isolated heterodimeric protein of any one of claims 1 to 4, wherein:
a) the first CH3-containing polypeptide is an antibody heavy chain; and/or
b) the second CH3-containing polypeptide is an antibody heavy chain; and/or
c) the heterodimeric protein further comprises one or more antibody light chains; and/or
d) the antibody light chain is a common antibody light chain.

6. A pharmaceutical composition comprising the isolated heterodimeric protein of any one of claims 1-5.

7. An isolated nucleic acid encoding the first and second human IgG CH3-containing polypeptides of any one of claims 1-5.

8. A recombinant host cell comprising the isolated nucleic acid of claim 7.

9. A method of producing the isolated heterodimeric protein of claims 1 to 5, comprising culturing the recombinant host cell of claim 8 under conditions that allow expression of the first and second human IgG CH3-containing polypeptides.

10. A method for improving stability of a heterodimeric protein that contains a first human IgG CH3-containing polypeptide which comprises the amino acid variant L351D and L368E, and a second human IgG CH3-containing polypeptide which comprises the amino acid variant T366K and L351K, the method comprising introducing an amino acid variant into the first human IgG CH3-containing polypeptide at position S364, K409 and/or K360.

11. A method for decreasing the stability of a homodimeric protein that contains a first human IgG CH3-containing polypeptide which comprises the amino acid variant L351D and L368E, the method comprising introducing an amino acid variant into the first human IgG CH3-containing polypeptide at position S364, K409 and/or K360.

12. A method for improving the yield of a heterodimeric protein comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, comprising introducing an amino acid variant into the first human IgG CH3-containing polypeptide at position S364, K409 and/or K360.

13. A method of increasing the purity of a heterodimeric protein comprising a first human IgG CH3-containing polypeptide and a second human IgG CH3-containing polypeptide, wherein said first human IgG CH3-containing polypeptide comprises the amino acid variant L351D and L368E and said second human IgG CH3-containing polypeptide comprises the amino acid variant T366K and L351K, comprising (a) introducing an amino acid variant into the first human IgG CH3-containing polypeptide at position S364, K409 and/or K360; and (b) subjecting the heterodimeric protein to ion exchange chromatography.

14. The method of any one of claims 10 to 13, wherein:
a) the 364 amino acid is valine, isoleucine, threonine, glutamine or leucine; and/or
b) the 409 amino acid is isoleucine, leucine or glutamate; and/or
c) the 360 amino acid is aspartate.

15. A heterodimeric protein produced by the method of claim 9.


SEQUENCE LISTING (17 sequences)

SEQ ID NO: 1 - DNA, 696 bp, Artificial Sequence
  MV1708_DE_S364V
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccaagaaccaggtcgtcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 2 - DNA, 696 bp, Artificial Sequence
  MV1708_DE_S364L
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccaagaaccaggtcctcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 3 - DNA, 696 bp, Artificial Sequence
  MV1708_DE_S364I
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccaagaaccaggtcatcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 4 - DNA, 696 bp, Artificial Sequence
  MV1708_DE_S364T
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccaagaaccaggtcacactgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 5 - DNA, 696 bp, Artificial Sequence
  MV1708_DE_S364Q
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccaagaaccaggtccagctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 6 - DNA, 696 bp, Artificial Sequence
  MV1708_DE_K409I
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcatcctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 7 - DNA, 696 bp, Artificial Sequence
  MV1708_DE_K409L
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcctgctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 8 - DNA, 696 bp, Artificial Sequence
  MV1708_DE_K409E
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcgaactcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 9 - DNA, 696 bp, Artificial Sequence
  CH2-3_DE_K360D-S364I
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccgacaaccaggtcatcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 10 - DNA, 696 bp, Artificial Sequence
  CH2-3_DE_K360D-S364T
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccaagaaccaggtcctcctgacctgcgaggtcaaaggcttctatcccagcgacatcgccgtggagtgggagagcaatgggcagccggagaacaactacaagaccacgcctcccgtgctggactccgacggctccttcttcctctatagcctgctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 11 - DNA, 696 bp, Artificial Sequence; identical to SEQ ID NO: 10
  CH2-3_DE_K360D-S364V

SEQ ID NO: 12 - DNA, 695 bp, Artificial Sequence
  CH2-3_DE_K360D-K409E
ccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccgacaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcgagctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 13 - DNA, 696 bp, Artificial Sequence
  CH2-3_DE_S364T-K409E
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccaagaaccaggtcacactgacctgcgaggtcaaaggcttctatcccagcgacatcgccgtggagtgggagagcaatgggcagccggagaacaactacaagaccacgcctcccgtgctggactccgacggctccttcttcctctatagcgagctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 14 - DNA, 696 bp, Artificial Sequence; identical to SEQ ID NO: 10
  CH2-3_DE_S364L-K409L

SEQ ID NO: 15 - DNA, 696 bp, Artificial Sequence
  MV1708_DE_K360D
tccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtttcgaacaaagccctcccagcccccatcgagaaaaccatctccaaagccaaagggcagccccgagaaccacaggtgtacaccgaccccccatcccgggaggagatgaccgacaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagtttaacggatcttaattaatccgagctcggtaccaagcttaag

SEQ ID NO: 16 - protein, 122 aa, Artificial Sequence
  VH sequence of anti-TT Fab
EVQLVETGGGVVQPGRSLRLSCAASGFTFSQYAMHWVRQAPGKGLEWVAIISHDERNKYYVDSGMGRFTISRDNSKNTLFLQMNSLRSEDTAVYYCARDMRKGGYYYGFDVWGQGTTVTVSS

SEQ ID NO: 17 - protein, 118 aa, Artificial Sequence
  VH sequence of anti-CD3 Fab
QVQLVESGGGVVQPGRSLRLSCAASGFTFRSYGMHWVRQAPGKGLEWVAIIWYSGSKKNYADSVKGRFTISRDNSKNTLYLQMNSLRAEDTAVYYCARGTGYNWFDPWGQGTLVTVSS